(19) United States
(12) Patent Application Publication
(10) Pub. No.: US 2016/0022727 A1
(43) Pub. Date: Jan. 28, 2016

(54) IMMUNOMODULATING COMPOUNDS AND RELATED COMPOSITIONS AND METHODS

(71) Applicants: CALIFORNIA INSTITUTE OF TECHNOLOGY, PASADENA, CA (US); BRIGHAM AND WOMEN'S HOSPITAL, BOSTON, MA (US)

(72) Inventors: June L. ROUND, PASADENA, CA (US); Sarkis K. MAZMANIAN, PORTER RANCH, CA (US); Ryan Michael O'CONNELL, PASADENA, CA (US); Dennis L. KASPER, CHARLESTOWN, MA (US)

(21) Appl. No.: 14/631,760

(22) Filed: Feb. 25, 2015

Related U.S. Application Data
(63) Continuation of application No. 12/831,131, filed on Jul. 6, 2010, now abandoned.
Application No. 12/831,131 is a continuation-in-part of application No. 12/267,602, filed on Nov. 9, 2008, now abandoned.
(60) Provisional application No. 61/223,294, filed on Jul. 6, 2009.
(60) Provisional application No. 61/355,025, filed on Jun. 15, 2010.
(60) Provisional application No. 61/002,705, filed on Nov. 9, 2007.
(60) Provisional application No. 61/008,407, filed on Dec. 20, 2007.
(60) Provisional application No. 61/196,046, filed on Oct. 14, 2008.

(51) Int. Cl. A61K 31/715

(52) U.S. Cl. CPC A61K 31/715

(57) ABSTRACT

Provided herein are compounds, compositions and methods for balancing a T-helper cell profile and in particular Th1, Th2, Th17 and Treg cell profiles, and related methods and compositions for treating or preventing an inflammatory condition associated with an imbalance of a T-helper cell profile.

### CROSS REFERENCE TO RELATED APPLICATIONS

This application is a continuation of U.S. patent application Ser. No. 12/831,131, filed on Jul. 6, 2010, which, in turn, claims priority to U.S. Provisional Application Ser. No. 61/223,294, entitled “PSA from Bacteroides Fragilis Cures Established Colitis” and filed on Jul. 6, 2009, and U.S. Provisional Application Ser. No. 61/355,025, entitled “PSA from Bacteroides Fragilis Cures Established Colitis” and filed on Jun. 15, 2010, the disclosure of all of which are incorporated by reference in their entirety. U.S. patent application Ser. No. 12/831,131 is also a continuation in part of U.S. patent application Ser. No. 12/267,602, entitled Immunomodulating Compounds and Related Compositions and Methods” and filed on Nov. 9, 2008, which, in turn, claims priority to U.S. Provisional Application No. 61/002,705, entitled “Host-Bacterial Mutualism by a Microbial Symbiosis Factor Prevents Inflammatory Disease” and filed on Nov. 9, 2007 (Docket No. CIT 5031-P), U.S. Provisional Application No. 61/008,407, entitled “Host-Bacterial Mutualism by a Microbial Symbiosis Factor Prevents Inflammatory Disease” and filed on Dec. 20, 2007 (Docket No. CIT 5031-P2) and U.S. Provisional Application No. 61/196,046, entitled “A Molecule from a Symbiotic Gut Bacteria Controls Systemic Inflammation” and filed on Oct. 14, 2008 (Docket No. CIT 5250-P), the disclosure of all of which are incorporated herein by reference in its entirety.

### STATEMENT OF GOVERNMENT GRANT

The U.S. Government has certain rights in this invention pursuant to Grant No. AI039576 and grant No. DK078938 awarded by the National Institutes of Health.

### FIELD

The present disclosure relates to the immune system, and, in particular, to an immunomodulating compound able to control T cell differentiation and/or cytokines production associated with an immunitary response in an individual.

### BACKGROUND

T cells belong to a group of white blood cells known as lymphocytes, and play a central role in cell-mediated immunity. In particular, T helper cells (also known as effector T cells or Th cells) are a sub-group of lymphocytes (a type of white blood cell or leukocyte) that plays an important role in establishing and maximizing the capabilities of the immune system and in particular in activating and directing other immune cells. More particularly, Th cells are essential in determining B cell antibody class switching, in the activation and growth of cytotoxic T cells, and in maximizing bactericidal activity of phagocytes such as macrophages.

Different types of Th cells have been identified that originate in outcome of a differentiation process and are associated with a specific phenotype. Following T cell development, matured, naïve (meaning they have never been exposed to the antigen to which they can respond) T cells leave the thymus and begin to spread throughout the body. Once the naïve T cells encounter antigens throughout the body, they can differentiate into a T-helper 1 (Th1), T-helper 2(Th2), T-helper 17 (Th17) or regulatory T cell (Treg) phenotype.

Each of these Th cell types secretes cytokines, proteins or peptides that stimulate or interact with other leukocytes, including Tₕ cells. However, each cell type has a peculiar phenotype and activity that interferes and often conflict with the other. Th1, Th2, and Th17 (inflammatory T-helper or inflammatory Th), promote inflammation responses trough secretion of pro-inflammatory cytokines, such as IL-1, IL-6, TNF-a, IL-17, IL21, IL23, and/or through activation and/or inhibition of other T cell including other Th cells (for example Th1 cell suppresses Th2 and Th17, Th2 suppresses Th1 and Th17). Tregs instead, are a component of the immune system that suppresses biological activities of other cells associated to an immune response. In particular, Tregs can secrete immunosuppressive cytokines TGF-beta and Interleukin 10, and are known to be able to limit or suppress inflammation.

An imbalance in the profile of any of the inflammatory T-helper cells is usually associated with a condition in an individual. For example, an increase profile for Th1 or Th17 leads to autoimmunity, whereas an increased Th2 cell profile leads to allergies and asthma. In particular, imbalance of Th17 cell profile has been associated with several autoimmunitary conditions. Treg cells suppress inflammation induced by all 3 other T cell lineages, and thus are crucial for preventing uncontrolled inflammation, which leads to disease. Therefore, a balanced T-helper profile is critical for health in individuals.

### SUMMARY

Provided herein, are immunomodulating compounds and related methods and compositions that are suitable to balance a T-helper cell profile, and in particular to balance the cell profile of at least one of Th1, Th2, Th17 and Treg cells in an individual. More particularly, provided herein are methods and compositions based on the surprising immunomodulating properties of PSA polysaccharide A (PSA) and other zwitterionic polysaccharides (ZPs) that make those polysaccharides suitable for treatment, prevention and control of inflammations and inflammatory conditions in an individual.

According to a first aspect, a method to balance a T-helper cell profile in an individual is disclosed. The method comprises administering to the individual an effective amount of a zwitterionic polysaccharide.

According to a second aspect, a method to balance a cell profile of at least one Th cell selected from the group consisting of Th1, Th2, Th17 and Treg, in an individual is disclosed. The method comprises administering to the individual an effective amount of a zwitterionic polysaccharide.

According to a third aspect, a method to control cytokine production in an individual, is disclosed, the cytokine being at least one of IL-1, IL-6, TNF-a, IL-17, IL21, IL23. The method comprises administering to the individual an effective amount of a zwitterionic polysaccharide.

According to a fourth aspect, a method to control inflammation associated with a Th− cell profile imbalance in an individual is disclosed. The method comprises administering to the individual an effective amount of a zwitterionic polysaccharide.

According to a fifth aspect, a method to treat or prevent conditions associated with an imbalanced cell profile of at least one Th cell selected from the group consisting of Th1, Th2, Th17 and Treg in an individual is disclosed. The method comprises administering to the individual an effective amount of a zwitterionic polysaccharide.

According to a sixth aspect, a method to treat or prevent conditions associated with production of at least one of IL-1, IL-6, TNF-a, IL-17, IL21, IL23 cytokines in an individual, is disclosed. The method comprises administering to the individual an effective amount of a zwitterionic polysaccharide.

According to a seventh aspect, an anti-inflammatory composition is disclosed. The anti-inflammatory composition comprises a zwitterionic polysaccharide and a suitable vehicle, wherein the zwitterionic polysaccharide is comprised in an amount of from about 1 to about 100 μg.

The compositions and methods herein disclosed can be used in several embodiments to simultaneously control and balance the profile of Th1, Th2, Th17 and Treg cells in an individual, thus preventing or treating conditions associated with an imbalanced profile for those cytokines in the individual.

The compositions and methods herein described can be used in connection with medical, pharmaceutical, veterinary applications as well as fundamental biological studies and various applications, identifiable by a skilled person upon reading of the present disclosure, wherein investigating the possible role of a zwitterionic polysaccharide is desirable.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description, serve to explain the principles and implementations of the disclosure.

FIG. 1 shows an exemplary ZP mediated protection from experimental colitis in individuals according to some embodiments herein disclosed. Panel (a) shows diagrams summarizing the results of mono-association of germ-free mice with wild-type B. fragilis and B. fragilis DPSA (mean percentages±Standard Deviation (SD) for 3 experiments: conventional, 38.4%±2.2; germ-free, 26.7%±1.3; B. fragilis, 40.8%±3.1; B. fragilis DPSA, 28.8%±2.6). All cells gated on CD4⁺ splenocytes. Panel (b) shows a diagram illustrating the results of co-colonization experiments of H. hepaticus with B. fragilis and B. fragilis DPSA (two-tailed p value, 0.004; Mann-Whitney U test). Combined data from 2 independent experiments are shown. Error bars show SD for triplicate samples. Panel (c) shows a diagram illustrating the results of ELISA test of colon organ cultures to detect TNFa levels in animals co-colonized with H. hepaticus and wild-type B. fragilis or B. fragilis DPSA. Panel (d) shows a diagram illustrating the results of a Q-PCR for IL-23p19 performed on splenocytes, normalized to L32 expression. Error bars show SD for triplicate samples.

FIG. 2 shows an exemplary ZP mediated cytokine control according to some embodiments herein disclosed. In particular, FIG. 2 shows diagrams illustrating the results of ELISA tests for the detection of the pro-inflammatory cytokines IL-12p40 (left) and IL-1b (right) in animals co-colonized with H. hepaticus and wild-type B. fragilis or B. fragilis DPSA over those in control animals (C57BL/6). Results are from one trial of 2 independent experiments. Error bars indicate SD values from studies of colons recovered from 4 animals per group.

FIG. 3 shows an example of ZP mediated control of TNFa expression by CD4⁺ T cells according to some embodiments herein disclosed. In particular FIG. 3 CD4⁺ cells were purified from pooled splenocytes from each group (4 mice per group) and restimulated in vitro with PMA and ionomycin in the presence of brefeldin A for 4 hours. Cells were stained for intracellular TNFα. Cells within the lymphocyte gate were included in the analysis, and numbers indicate the percentage of cells producing TNFα. Purified cells were >90% CD4⁺. Animals colonized with PSA-producing B. fragilis during protection displayed lower TNFa levels than diseased animals.

FIG. 4 shows control experiments supporting various embodiments herein described. Panel (a) shows an ethidium bromide-stained gel electrophoresis of H. hepaticus-specific Q-PCR performed following co-colonization with wild-type and various mutants of B. fragilis after 8 weeks. M: Marker. 1: Rag2^{-/-} animals with CD4⁺CD45Rb^{high} T cell transfer colonized with H. hepaticus alone. 2: Rag2^{-/-} animals with CD4⁺CD45Rb^{high} T cell transfer colonized with H. hepaticus and B. fragilis 9343 (wt). 3: Rag2^{-/-} animals with CD4⁺CD45Rb^{high} T cell transfer colonized with H. hepaticus and B. fragilis DPSA. 4: C57BL/6 mice colonized with H. hepaticus alone. Note: H. hepaticus readily colonized animals but did not induce disease (FIG. 1). Primers for H. hepaticus 16S rDNA: (HB-15) 5′-GAAACTGTTACTCTG-3′ (SEQ ID NO: 1) and (HB-17) 5′-TCAAGCTCCCCGAAGGG-3′ (SEQ ID NO: 2). Panel (b) shows ethidium bromide-stained gel electrophoresis of B. fragilis-specific Q-PCR performed following co-colonization with wild-type and various mutants of B. fragilis after 8 weeks. A: Rag2^{-/-} animals with CD4⁺CD45Rb^{high} T cell transfer colonized with H. hepaticus and B. fragilis 9343 (wt). B: Rag2^{-/-} animals with CD4⁺CD45Rb^{high} T cell transfer colonized with H. hepaticus and B. fragilis DPSA. C: Rag2^{-/-} animals with CD4⁺CD45Rb^{high} T cell transfer colonized with H. hepaticus alone. D: C57BL/6 mice colonized with H. hepaticus alone. E: B. fragilis genomic DNA (positive control). M: Marker. Primers for B. fragilis ssr3 (finB) gene: (ssr3-F) 5′-TATTTGCGAGAAGGTGAT-3′ (SEQ ID NO: 3) and (ssr3-r) 5′-TAAACGCTTTGCTGCTAT-3′ (SEQ ID NO: 4).

FIG. 5 effects associated to a ZP-mediated protection according to some embodiments herein disclosed. In particular, FIG. 5 shows a diagram illustrating the results of Q-PCR experiments directed to quantitate H. hepaticus in animals co-colonized with H. hepaticus and wild-type B. fragilis or B. fragilis DPSA. The results was assessed according to Young et al., 2004¹ as log¹⁰ number of copies of a known gene (cytolethal distending toxin). Animals contained equivalent levels of H. hepaticus at the end of the experiment.

FIG. 6 shows a ZPs mediated protection according to some embodiments herein disclosed. Panel (a) shows a diagram illustrating the results of a colonization with H. hepaticus in absence (second column) or in presence of purified PSA (third column) (Kruskal-Wallis comparisons of all groups: p>0.05 for dissimilar results, p<0.01 for similar results; Mann-Whitney U test: two-tailed p value, 0.0002). Panel (b) shows a diagram illustrating results of experiments directed to detect wasting disease in Rag2^{-/-} animals following transfer of CD4⁺CD45Rb^{high} T cells and colonization with H. hepaticus (PBS+Hh) in presence or absence of PSA as indicated. ANOVA indicates that comparisons between all indicated groups (asterisks) are statistically significant. Panel (c) shows the architecture of colonic sections from wild-type animals (left panel); following transfer of CD4⁺CD45Rb^{high} T cell into Helicobacter-colonized Rag2^{-/-} mice (middle panel); oral PSA treatment of Helicobacter-colonized animals (right panel). Images in each row are the same magnification.

FIG. 7 shows a ZP modulated immune response according to some embodiments herein disclosed. Panel (a) shows a diagram illustrating the correlation between oral PSA administration and body weight related to TNBS-treated PBS controls. ANOVA values for all indicated groups (asterisks) are statistically significant. Error bars show SD between 4 animals per group. Panel (b) shows colon sections from TNBS+PBS-treated groups, from TNBS+PSA-treated animals and from a control (representative sections from animals in 2 independent experiments). Panels (c, d) show diagram illustrating the results of Q-PCR of purified CD4⁺ T cells from MLNs with IL-17A (Panel c) and TNFa (Panel d) in presence or absence of PSA during disease. Error bars are from duplicate runs of 3 independent experiments. Panels (e, f) show diagrams illustrating transcriptional expression of IL17A (Panel e) and TNFα (Panel f) from homogenized colons of TNBS+PBS-treated groups, from TNBS+PSA-treated animals and from a control. Error bars are from duplicate runs of 3 independent experiments.

FIG. 8 shows a ZP mediated control of cytokine expression according to some embodiments herein disclosed. Panel (a) shows a diagram illustrating the results of Q-PCR assay of colons for IL-10 in wild type mice treated with ethanol (control), TNBS, or TNBS and PSA. Error bars show SD for triplicate samples. Panel (b) shows a diagram illustrating Q-PCR results for IL-10 expression in CD4⁺ T cells purified from MLNs of TNBS-treated groups. Error bars show SD for triplicate samples. Panel (c) shows a diagram illustrating the effects of incubation of BMDC/T cell co-cultures with purified PSA LPS and a-CD3/a-CD28 on IL-10 production. Error bars show SD for triplicate samples. Panel (d) shows a diagram illustrating the results of an infection of BMDC-T cell co-cultures with increasing concentrations of H. hepaticus (multiplicity of infection: 0.1, 1.0, and 10, as depicted by triangles) on TNFa release in presence (middle three bars) or absence (left three bars) of PSA and following the addition of aIL-10R right three bars □. Error bars show SD values of experiments run in triplicate.

FIG. 9 shows a ZP mediated control of cytokine expression according to some embodiments herein disclosed. In particular, FIG. 9 shows a diagram illustrating the results for an IL-10 ELISA of supernatants of primary BMDC-T cell co-cultures incubated for 48 hours with H. hepaticus alone or with H. hepaticus and B. fragilis (wild-type or APSA) at a multiplicity of infection of 5. Error bars show SD values for samples run in duplicate and represent 3 independent experiments.

FIG. 10 shows a ZP mediated control of cytokine expression according to some embodiments herein disclosed. In particular, FIG. 10 shows a diagram illustrating the results of an infection of BMDC-T cell co-cultures with increasing concentrations of live H. hepaticus (multiplicity of infection: 0.1, 1.0, and 10, as depicted by triangles) on release of the cytokine IL-1b in presence (middle three bars) or absence (left three bars) of PSA and following the addition of aIL-10R right three bars. Error bars show SD values for experiments run in triplicate.

FIG. 11 shows a ZP mediated protection from inflammation according to some embodiments herein disclosed. Panels (a, b) show diagrams illustrating results of ELISA detection for pro-inflammatory cytokines TNFα (Panel a) and IL-17A (Panel b) in IL-10^{-/-} mice left uncolonized (control) or colonized with H. hepaticus (to induce inflammation) either alone or in combination with B. fragilis (wild-type or APSA). Error bars show SD for triplicate samples. Panel (c) shows a diagram illustrating the colitis scores in Rag^{-/-} animals with CD4⁺CD45Rb^{high} T cell transfer colonized with H. hepaticus with or without PSA and in presence of neutralizing antibodies to IL-10 block (α-IL1OR). Data represent 2 independent experiments. Panel (d) shows a diagram illustrating colitis scores in Rag^{-/-} animals with CD4⁺CD45Rb^{high} T cell transferred from IL-10^{-/-} mice colonized with H. hepaticus with PSA or PBS. Results are shown for 1 representative trial of 2 independent experiments. Panel (e) shows histologic colon sections Rag^{-/-} animals with CD4⁺CD45Rb^{high} T cell transferred from IL-10^{-/-} mice colonized with H. hepaticus with PSA or PBS. All images are the same magnification. Panel (f) shows a diagram illustrating the mean body weights for groups of Rag^{-/-} animals (n=4) with CD4⁺CD45Rb^{high} T cell transferred from IL-10^{-/-} mice colonized with H. hepaticus with PSA or PBS.

FIG. 12 shows effect of a ZP administration supporting embodiments herein disclosed. In particular, FIG. 12 shows a diagram illustrating the variation on body weight in groups of 4 C57BL/6 mice treated with PSA (or PBS) and then subjected to rectal administration of TNBS or vehicle (control). Mean body weights (shown as percentages of initial weight) are shown for each group; SD values indicate that, in the absence of IL-10, PSA cannot restore TNBS-induced weight loss. ANOVA demonstrates that weight loss in both TNBS-treated groups is statistically different from that in control animals.

FIG. 13 shows effects of a ZP administration supporting some embodiments herein disclosed. In particular, FIG. 13 shows results of histologic analysis of H&E-stained sections from a representative animal of groups of 4 C57BL/6 mice treated with PSA (or PBS) and then subjected to rectal administration of TNBS or vehicle (control). Results represent 2 independent experiments.

FIG. 14 shows inhibition of inflammation within extra-intestinal immune compartments following oral administration of ZPS according to some embodiments herein disclosed. In particular, Panel (a) shows a diagram illustrating the colonic histological score detected in untreated mice (control) and in mice treated with TNBS or TNBS/PSA. Each dot represents an individual mouse and the line indicates the average score of the group. Panel (b) shows a diagram illustrating the percent of survival in time of Balb/c mice undergoing TNBS induced colitis. n=16 mice in each group. Panel (c) shows an image of the spleen of untreated mice (control) and mice treated with TNBS or TNBS/PSA Panel (d) shows a diagram illustrating the relative units of TNF-α, IL-6, IL-17A and IL-10 within CD4+ splenocytes in untreated mice (control) and in mice treated with TNBS or TNBS/PSA. These data are representative of three independent experiments.

FIG. 15 shows protection from TNBS induced intestinal colitis following administration of ZPS to extra-intestinal sites according to some embodiments herein disclosed. In particular, Panel (a) shows a diagram illustrating the percent survival of mice undergoing TNBS induced colitis. n=10 mice in each group. Panel (b) shows a diagram illustrating variation of the spleen weight in untreated mice (Etoh) and in mice treated with TNBS or TNBS/PSA systemically administered. The weight of the spleen was used as an indicator of size. Each diamond represents the weight of the spleen from an individual animal. The bar indicates the average weight of the group. P values were determined by students T test.

FIG. 16 shows inhibition of inflammatory cytokines at both intestinal and systemic immune compartments following systemic administration of ZPS during TNBS induced colitis according to some embodiments herein disclosed. Panel (a) shows a diagram illustrating TNF-α production in CD4+T lymphocytes residing within the mesenteric lymph nodes (MLN) splenocytes in untreated mice (control) and in mice treated with TNBS or TNBS/PSA. Cells were collected from the MLN and stained with antibodies recognizing CD4 or TNF-a. Numbers within quadrants represent the percentage of cells. Panel (b) shows a diagram illustrating analysis of the expression of IL-12, IL-23, and IL-17 in colon of untreated mice (control) and mice treated with TNBS or TNBS/PSA. Panel (c) show a diagram illustrating TNF-α production in CD4+ T lymphocytes residing within the spleen of untreated mice (control) and of mice treated with TNBS or TNBS/PSA. Numbers within quadrants represent the percentage of cells. Panel (d) a diagram illustrating analysis of the expression of IL-12, IL-6, and IL-17 in spleen of untreated mice (control) and mice treated with TNBS or TNBS/PSA.

FIG. 17 shows inhibition of inflammation and death associated with systemic septic shock following administration of ZPS according to some embodiments herein disclosed. Panel (a): shows a diagram illustrating TNF-a serum levels in mice 1 and 4 hours post-administration of 100 μg of LPS alone. Mice were either pre-treated with PBS or 50 ug of PSA three times every other day before LPS administration. * indicates statistical significance as determined by a students t test. SD was determined from the serum of individual mice. These data are representative of three independent experiments. Panel (b): shows a diagram illustrating IL-6 serum levels in mice 1 and 4 hours post-administration of 100 μg of LPS. Pre-treatment as in panel a * indicates statistical significance as determined by a students t test. SD was determined from the serum of individual mice. These data are representative of three independent experiments. Panel (c): shows a diagram illustrating variation of the spleen weight in untreated mice (con) and in mice administered LPS within the intraperiotenal cavity (LPS) and pre-treated with PBS or PSA as in panel a. Each dot represents the weight of the spleen from an individual mouse. P values were determined by a students T test. Panel (d): shows a diagram illustrating the survival rate of animals undergoing septic shock induced by high dose (500 μg) administration of LPS and pre-treated with PSA or PBS. N=12 mice in each group. Panel (e): shows a diagram illustrating the serum concentrations of TNF-α in mice post-administration of 500₁₄ of LPS alone or pre-treated with PSA or PBS. p values were determined by students T test. Each dot represents an individual mouse. Panel (f) shows a diagram illustrating the serum concentrations of IL-6 in mice post-administration of 500 μg of LPS alone and pre-treated with PSA or PBS. p values were determined by students T test. Each dot represents an individual mouse.

FIG. 18 shows inhibition of inflammation and death associated with systemic septic shock following administration of ZPS according to some embodiments herein disclosed. Panel (a): shows a diagram illustrating TNF-a serum levels in mice pre-treated with PBS or PSA and administered LPS. Serum was collected 1 and 4 hours post-administration of LPS in IL-10⁻/⁻ mice. * indicates statistical significance as determined by a students t test. SD was determined from the serum of individual mice. Panel (b): shows a diagram illustrating IL-6 serum level in mice pre-treated with PSA or PBS. Serum was collected 1 and 4 hours post-administration of LPS in IL-10^{-/-} mice. * indicates statistical significance as determined by a students t test. SD was determined from the serum of individual mice Panel (c): shows a diagram illustrating percent survival in mice post-administration of LPS alone or together with PSA in IL-10⁻/⁻ mice. N=8 mice in each group.

FIG. 19 shows a diagram illustrating additional effects of ZPS administration on inflamed tissues according to some embodiments herein disclosed.

FIG. 20 shows a diagram illustrating percent survival in mice treated with PSA before and after colitis induction compared to a control according to some embodiments herein described. In particular, 8 week old Balb/c mice were orally gavaged with 50 μg of purified PSA three times every other day prior to TNBS induction and every other day throughout the course of disease (triangle-PSA pre) or orally gavaged with 50 μg of PSA 24 hours post-TNBS induction and every other day throughout the course of disease (X). TNBS colitis was induced by rectal administration of 100 ml of 1.25% TNBS in 50% ethanol. Control mice were rectally administered 50% ethanol as a vehicle control (diamonds).

FIG. 21 shows a diagram illustrating changes in weight of mice treated with PSA before and after colitis induction compared to a control according to some embodiments herein described. Vehicle treated mice, no TNBS-diamond, PBS treated-with TNBS induction-squares, PSA treatment prior to TNBS induction-triangles, PSA treatment after TNBS induction-circles. Mice were weighed on the day of TNBS induction (day 0) and every day thereafter at the same time of day. * indicates a statistical difference between groups.

FIG. 22 shows a diagram illustrating results of a histological analysis performed in mice treated with PSA and/or TBS according to some embodiments herein described. In particular, the diagram of FIG. 22 shows that the colitis score by a blinded pathologist in sections of the colon of mice treated with TNBS only, or with PSA before or after TNBS treatment, as well as of control mice.

FIG. 23 shows a diagram illustrating results showing the percentage of Foxp3 in a CD4+CD25+Treg subset in mice treated with PSA before and after colitis induction compared to a control according to some embodiments herein described.

FIG. 24 shows diagrams illustrating results showing PSA ability to induce functional Foxp3+Tregs with an inducible phenotype according to some embodiments herein described. Panel A shows results of experiments in which Foxp3-GFP mice were orally gavaged with purified PSA, MLNs harvested and cells stained for CD4 and CD25. Each symbol represents the % of CD4+Foxp3-GFP+ cells from a single mouse. Results are representative of 3 independent trials with 4 mice per group. **p value of <0.01. Panel B shows results of experiments in which CD4+CD25+ cells were purified from the MLNs of mice treated with either PBS or PSA as indicated in A and incubated with CFSE-pulsed CD4+CD25− effector T cells in an in vitro suppression assay. Numbers indicate the percentage of cells undergoing at least one cellular division at 3 different ratios of effector T cells (Teff) and regulatory T cells (Treg). These data are representative of 2 independent trials. Panel C shows results of experiments in which Foxp3-GFP mice were orally treated with purified PSA. MLNs were extracted and CD4+Foxp3+ or the CD4+Foxp3− T cells were purified based on ±GFP expression (purity >99%). RNA was extracted and used for qRT-PCR. These data are representative of three independent experiments. Light bars indicated cells derived from PBS treated mice and dark bars from PSA-treated mice. Panel D shows results of experiments in which TLR2−/− mice were orally treated with PSA. MLNs were extracted and analyzed for the percentage of CD4+Foxp3+ cells. Each symbol in the bar graph represents the percentage of CD4+Foxp3+ from an individual mouse. NS, not significant. Results are representative of 3 independent trials with at least 3 mice per group. Panel E shows results of experiments in which CD4+CD25hi+ and CD4+CD25− T cell populations were FACS purified from MLNs of PSA-treated TLR2−/− mice. IL-10 levels were analyzed by qRT-PCR. Light and dark bars indicate IL-10 levels in PBS or PSA treated animals, respectively. Error bars represent the SD from samples of the same experiment run in triplicate. Results are representative of 2 independent trials with 4 mice per group.

FIG. 25 shows diagrams illustrating results showing PSA's ability to up-regulates the expression of genes for ICOS and Perforin according to some embodiments herein described. Foxp3-GFP mice were orally treated with purified PSA every other day for 6 days. MLNs were extracted and CD4+Foxp3+ or the CD4+Foxp3− T cells were purified by FACS based on ±GFP expression (purity >99%). RNA was extracted and used for qRT-PCR. These data are representative of three independent experiments. Light bars indicated cells derived from PBS treated mice and dark bars from PSA-treated mice. Error bars represent SD from samples run in triplicate from a single experiment. These results represent three independent trials.

FIG. 26 shows a diagram illustrating results showing IFN-γ levels in the MLNS in presence or absence of PSA according to some embodiments herein described. Cells were taken from the MLN and stained with CD4, Foxp3 and IFN-γ. Plots are gated on CD4+ cells and numbers rounded to the nearest tenth. These are data are representative of three independent trials.

FIG. 27 shows a diagram illustrating results showing IFN-γ levels in spleen in presence or absence of PSA according to some embodiments herein described. GF Rag−/− animals were colonized with B. fragilis or B. fragilis DPSA and CD4+Foxp3− T cell from Foxp3-GFP mice were adoptively transferred. After 3 weeks, spleens were harvested and CD4+T cells stained for IL-10 and IFNγ after restimulation with PMA/Ionomycin in the presence of brefeldin A. Plots are gated on CD4+Foxp3− (GFP−) cells. Numbers are rounded to the nearest tenth.

FIG. 28 shows diagrams illustrating results showing PSA ability to expand functional Foxp3+Tregs during protection from experimental colitis according to some embodiments herein described. Panel A shows results of experiments in which Balb/c mice were treated with PSA or PBS during TNBS induced colitis and analyzed for the percentage of CD25+Foxp3+ cells within the CD4+ population of the MLN. These data are representative of 3 independent trials with 4 mice per group. Panel B shows results of experiments in which mice were treated as in A, MLN cells were counted and absolute numbers of CD4+CD25+Foxp3+ cells determined (×10³). Numbers represent the average of 4 mice with error bars indicting SD, and are representative of 3 independent trials. Panel C shows results of experiments in which RNA was extracted from the MLNs of mice treated with PSA and foxp3 transcript expression is shown, normalized to β-actin expression in the total lymph node. Numbers represent the average of 4 mice with error bars indicating SD, and are representative of 3 independent trials. Panel D shows results of experiments in which CD4+CD25+ cells were purified from the MLNs of colitic PBS or PSA-treated mice and incubated with CFSE-pulsed CD4+CD25− responder cells in an in vitro suppression assay. Numbers indicate the percentage of cells undergoing at least one cellular division at 2 different ratios of effector T cells (Teff) and regulatory T cells (Treg). These data are representative of 2 independent trials Panel E shows results of experiments in which C57B1/6 WT or TLR2−/− animals were gavaged with purified PSA (or PBS) prior to TNBS administration. Colons were fixed, sectioned and H&E stained. Representative histological sections are shown. Panel F shows results of experiments in which Colitis scores show that PSA protects WT but not TLR2−/− animals from experimental colitis. Each symbol represents a separate animal, and data are from 2 independent trails. *p value of <0.05. NS, not significant. Panel G shows results of experiments in which RNA was extracted from the MLNs of indicated mice either treated with PBS or PSA, and the expression levels of IL-10 and IL-17A were assayed by qRT-PCR. Relative units are represented as transcript levels relative to expression of a housekeeping gene (L32). Results are representative of 3 independent trials with 4 mice per group; error bars indicate SD. Panel H shows results of experiments in which Percentages of CD4+CD25+Foxp3+ T cells in the MLNs of TLR2−/− mice treated with PBS or PSA during TNBS induced colitis. Plots are gated on CD4+T cells. Results are representative of 3 independent trials with 4 mice per group.

FIG. 29 shows a diagram illustrating results showing PSA's ability to expand Tregs, but not B cells during experimental colitis according to some embodiments herein described. The MLN of TNBS mice were analyzed for the presence of B cells by FC staining for B220 and CD19. These results are representative of three independent experiments.

FIG. 30 shows a diagram illustrating results showing PSA's ability to increase the amount of Foxp3 protein during TNBS according to some embodiments herein described. Cells from the MLN were extracted from indicated mice and stained with CD4 and Foxp3. Plots are gated on CD4+ cells. The histogram indicates that PSA treated animals have a higher expression of Foxp3 than cells from TNBS animals. These data are representative of three independent experiments.

FIG. 31 shows diagrams illustrating results showing PSA's ability to treat animals with colitis according to some embodiments herein described. Panel A shows results of experiments in which Balb/c mice were either pre-treated with PSA prior to administration of 1% TNBS (pre) or orally treated with PSA 24 hours after the administration of TNBS (post). Mice were weighed daily for eight days. *p value of <0.05 at the indicated time points. Panels B and C shows results of experiments in which Colons were extracted from groups of mice treated as in A, fixed, sectioned and stained with H&E to determine disease severity 5 days post-TNBS administration. Representative sections are shown in Panel B. Colitis scores are shown in Panel C. Data are representative of three independent experiments, p values determined by a two-tailed Mann-Whitney U-test. **p value of <0.01. Panel D shows results of experiments in which MLNs from indicated mice were extracted and cells stained with antibodies to detect for CD4, CD25 and Foxp3. Numbers indicate the percentage of CD25+Foxp3+ cells within CD4+ population of cells. Results are representative of 3 independent trials with 4 mice per group; error bars indicate SD. Panel E shows results of experiments in which Balb/c mice were treated with PSA as indicated in A and rectally administered 2% TNBS. Number of animals: 8 mice in the control group, 7 in the pre-TNBS PSA group and 5 in the post-TNBS PSA groups and 11 in the TNBS+PBS group. Data in E are the combination of 2 independent experiments.

FIG. 32 shows diagrams illustrating results showing that PSA treatment of animals after induction of TNBS colitis according to some embodiments herein described up-regulates of IL-10 and Foxp3 expression. Balb/c animals were induced for TNBS colitis and subsequently given PBS 1 day post TNBS induction (TNBS+PBS) or PSA 1 or 2 days post-TNBS induction (post-TNBS PSA 1 or 2). Five days post-TNBS treatment RNA was extracted from the colon and IL-10 and Foxp3 gene expression was assayed by qRT-PCR. Error bars represent the SD from samples run in triplicate from one independent trial.

FIG. 33 shows a diagram illustrating results showing that PSA treatment of colitic animals according to some embodiments herein described results in a decreased expression of inflammatory IL-17A. RNA was extracted from the MLN of indicated animals and expression levels of IL-17A were determined by qRT-PCR. Error bars indicate SD from four independent mice. *p value of <0.05. These data are representative of three independent trials.

FIG. 34 shows a diagram illustrating results showing that PSA's administration according to some embodiment herein described can decrease IL-6 in the serum. High doses of LPS (500 μg) were administered i.p. to Balb/c mice. 10 or 30 minutes post LPS administration, PSA was given retro-orbitally. 1 hour post-LPS, serum was collected and levels of IL-6 were determined.

FIG. 35 shows a diagram illustrating results showing that PSA's administration according to some embodiment herein described can decrease TNF-α in the serum. High doses of LPS (500 μg) were administered i.p. to Balb/c mice. 10 or 30 minutes post LPS administration, PSA was given retro-orbitally. 1 hour post-LPS, serum was collected and levels of TNF-α were determined.

FIG. 36 shows a diagram illustrating results showing that PSA's administration according to some embodiment herein described can increase survival of mice already suffering from endotoxic shock.

### DETAILED DESCRIPTION

Methods and compositions are herein disclosed that allow balancing a T-helper cell profile in an individual, based on the use of PSA or another zwitterionic polysaccharide.

The term “T-helper” as used herein with reference to cells indicates a sub-group of lymphocytes (a type of white blood cell or leukocyte) including different cell types identifiable by a skilled person. In particular, T-helper cell according to the present disclosure include effector Tₕ cells (such as Th1, Th2 and Th17)—i.e. Th cells that secrete cytokines, proteins or peptides that stimulate or interact with other leukocytes, including Tₕ cells—and suppressor Th cells (such as Treg) i.e. Th cells that suppress activation of the immune system and thereby maintain immune system homeostasis and tolerance to self-antigens. Mature Tₕ cells are believed to always express the surface protein CD4. T cells expressing CD4 are also known as CD4⁺ T cells. CD4⁺ T cells are generally treated as having a pre-defined role as helper T cells within the immune system, although there are known rare exceptions. For example, there are sub-groups of suppressor T cells, natural killer T cells, and cytotoxic T cells that are known to express CD4 (although cytotoxic examples have been observed in extremely low numbers in specific disease states, they are usually considered non-existent).

The term “cell profile as used herein indicates a detectable set of data portraying the characterizing features of a cell that distinguish the characterized cell from another. In particular, when referred to a T helper cell, the wording “cell profile” indicates a detectable set of data related to a marker cytokine that is produced by the Th cell and characterizes the Th cell with respect to another. For example, marker cytokine for Th1 cell is Interferon-g; marker cytokine for Th2 is IL-4, marker cytokine for Th7 is 11-17 and marker cytokine for Treg is IL-10. Accordingly, the wording “Th17 cell profile” as used herein indicates the detectable set of data, such as presence and amount, related to production of IL-17 in a certain organ or tissue of the individual wherein the presence and/or activity of Th1 cell is investigated. Similar definitions apply to the other Th cell types. On the other hand, when the wording “cell profile” is referred to a subset of Th cell including more then one Th cell type, the wording “T-helper cell profile” indicates a detectable set of data related to each marker cytokine that is produced by and characterizes each, of the T-helper cells of the subset.

The term “balance” as used herein with reference to a “Th cell profile” as used herein indicates the activity of bringing the cell profile to a status associated with absence of an inflammatory response. Similarly the term “balanced Th profile” indicates the Th cell profile status associated with absence of an inflammatory response and in particular to the detectable set of data related to a marker cytokine that is produced by the T helper cell and characterizes the T helper cell with respect to another in absence of an inflammatory response. When the term “T-helper cell” profile refers to a subset of Th cell including more then one Th cell type, the term “balanced Th profile” refers instead to the relative ratio between the detectable set of data related to each marker cytokine that is produced by and characterizes each, of the T-helper cells. For example, a “balanced Th cell profile” referred to a Th cells subset comprising Th1, Th2 and Th17 indicates the relative ratio of data related to Interferon-gamma, IL-4 and IL17 associated with absence of an inflammatory response.

The term “zwitterionic polysaccharide” as used herein indicates synthetic or natural polymers comprising one or more monosaccharides joined together by glicosidic bonds, and including at least one positively charged moiety and at least one negatively charged moiety. Zwitterionic polysaccharides include but are not limited to polymers of any length, from a mono- or di-saccharide polymer to polymers including hundreds or thousands of monosaccharides. In some embodiments, a zwitterionic polysaccharide can include repeating units wherein each repeating unit includes from two to ten monosaccharides, a positively charged moiety (e.g. an free positively charged amino moiety) and a negatively charged moiety (such as sulfonate, sulfate, phosphate and phosphonate). In some embodiment ZPs can have a molecular weight comprised between 500 Da and 2,000,000 Da. In some embodiments, the ZPs can have a molecular weight comprised between 200 and 2500. Exemplary ZPS include but are not limited to PSA and PSB from Bacteroides Fragilis, CP5/CD8 from Staphylococcus aureus, and Sp1/CP1 from Streptococcus pneumonia. Zwitterionic polysaccharides can be isolated from natural sources, and in particular from bacterial sources, e.g. by purification. Zwitterionic polysaccharides can also be produced by chemical or biochemical methods, as well as by recombinant microorganism technologies all identifiable by a skilled person. Thus, those methods and technologies will not be further described herein in detail.

The wording “polysaccharide A” as used herein indicates a molecule produced by the PSA locus of Bacteroides Fragilis and derivatives thereof which include but are not limited to polymers of the repeating unit {→+3)α-d-AATGalp(1→4)[β-d-Galf(1→3)]-d-GalpNAc(1 3)β-d-Galp(1→}, where AATGal is acetamido-amino-2,4,6-trideoxygalactose, and the galactopyranosyl residue is modified by a pyruvate substituent spanning O-4 and O-6. The term “derivative” as used herein with reference to a first polysaccharide (e.g., PSA), indicates a second polysaccharide that is structurally related to the first polysaccharide and is derivable from the first polysaccharide by a modification that introduces a feature that is not present in the first polysaccharide while retaining functional properties of the first polysaccharide. Accordingly, a derivative polysaccharide of PSA, usually differs from the original polysaccharide by modification of the repeating units or of the saccharidic component of one or more of the repeating units that might or might not be associated with an additional function not present in the original polysaccharide. A derivative polysaccharide of PSA retains however one or more functional activities that are herein described in connection with PSA in association with the anti-inflammatory activity of PSA.

In some embodiments, the zwitterionic polysaccharide can be PSA and/or PSB. In some embodiments, the effective amount of ZP and in particular PSA and/or PSB is from about 1-100 micrograms to about 25 grams of body weight and the T-helper cell profile is balanced by balancing at least one of Th1, Th2, Th17 and Treg, in particular at least one of Th1, Th 2 and Treg and Th17. More particularly, in some embodiments, balance Th cell profile can be performed by balancing the Th17 cell profile.

In some embodiments, a ZP can be used to control cytokine production associated with inflammation in an individual. In particular, in some embodiments, ZPs can be administered to inhibit production of pro-inflammatory cytokine molecules such as TNF-a, IL1 or IL-6, IL21, IL23 and IL17.

The term “control” as used herein indicates the activity of affecting and in particular inhibiting a biological reaction or process, which include but are not limited to biological and in particular biochemical events occurring in a biological system, such as an organism (e.g. animal, plant, fungus, or micro-organism) or a portion thereof (e.g. a cell, a tissue, an organ, an apparatus).

The terms “inhibiting” and “inhibit”, as used herein indicate the activity of decreasing the biological reaction or process. Accordingly, a substance “inhibits” a certain biological reaction or process if it is capable of decreasing that biological reaction or process by interfering with said reaction or process. For example, a substance can inhibit a certain biological reaction or process by reducing or suppressing the activity of another substance (e.g. an enzyme) associated to the biological reaction or process, e.g. by binding, (in some cases specifically), said other substance. Inhibition of the biological reaction or process can be detected by detection of an analyte associated with the biological reaction or process. The term “detect” or “detection” as used herein indicates the determination of the existence, presence or fact of an analyte or related signal in a limited portion of space, including but not limited to a sample, a reaction mixture, a molecular complex and a substrate. A detection is “quantitative” when it refers, relates to, or involves the measurement of quantity or amount of the analyte or related signal (also referred as quantitation), which includes but is not limited to any analysis designed to determine the amounts or proportions of the analyte or related signal. A detection is “qualitative” when it refers, relates to, or involves identification of a quality or kind of the analyte or related signal in terms of relative abundance to another analyte or related signal, which is not quantified.

The term “cytokine” as used herein indicates a category of signaling proteins and glycoproteins extensively used in cellular communication that are produced by a wide variety of hematopoietic and non-hematopoietic cell types and can have autocrine, paracrine and endocrine effects, sometimes strongly dependent on the presence of other chemicals. The cytokine family consists mainly of smaller, water-soluble proteins and glycoproteins with a mass between 8 and 30 kDa. Cytokines are critical to the development and functioning of both the innate and adaptive immune response. They are often secreted by immune cells that have encountered a pathogen, thereby activating and recruiting further immune cells to increase the system's response to the pathogen.

Detection of inhibition of cytokine production can be performed by methods known to a skilled person including but not limited to ELISA, Q-PCR and intracellular cytokine staining detected by FACs and any other methods identifiable by a skilled person upon reading of the present disclosure.

In some embodiments, a ZP can be administered to inhibit production of at least one of TNF-a, IL-6, IL-17, IL-21 and IL-23. In particular, in some of those embodiments ZP can be administered systemically and in particular, orally, subcutaneously, intra peritoneally, and intravenously. In some embodiments ZP can be administered in an amount between about 1 and about 100 micrograms/25 grams of body weight.

Methods and compositions are herein disclosed that allow control of an inflammation associated with an imbalanced Th cell profile and or to production of at least one of the pro-inflammatory cytokines IL-1, IL-6, TNF-a, IL-17, IL21, IL23, and TGF-β in an individual.

The term “inflammation” and “inflammatory response as used herein indicate the complex biological response of vascular tissues of an individual to harmful stimuli, such as pathogens, damaged cells, or irritants, and includes secretion of cytokines and more particularly of pro-inflammatory cytokine, i.e. cytokines which are produced predominantly by activated immune cells such as microglia and are involved in the amplification of inflammatory reactions. Exemplary pro-inflammatory cytokines include but are not limited to IL-1, IL-6, TNF-a, IL-17, IL21, IL23, and TGF-β. Exemplary inflammations include acute inflammation and chronic inflammation. The wording “acute inflammation” as used herein indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). The wording “chronic inflammation” as used herein indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis. An inflammation can be controlled in the sense of the present disclosure by affecting and in particular inhibiting anyone of the events that form the complex biological response associated with an inflammation in an individual. In particular, in some embodiments, an inflammation can be controlled by affecting and in particular inhibiting cytokine production, and more particularly production of pro-inflammatory cytokines, following administration of a zwitterionic polysaccharide.

More particularly, in some embodiments, a ZP can be used to control an inflammation associated with IL-1, IL-6, TNF-a, IL-17, IL21, IL23, and/or TGF-β mediated inflammation in an individual. The wording “cytokine mediated inflammation” as used herein indicates an inflammation wherein the complex biological response to a harmful stimulus is controlled by cytokine molecules, such as pro-inflammatory cytokine molecules (e.g. TNF-a, IL1 and/or IL-6) and anti-inflammatory cytokine molecules (e.g. IL-10). Exemplary cytokine mediated inflammation include but are not limited to conditions mediated by IL-1, IL-6, TNF-α, IL-12p35, IL-17A, IL-21, IL-22, IFN-γ and/or IL-23p19.

In some embodiments, the cytokine is at least one of TNF-a, IL-17, IL-21, and IL-23 and the cytokine mediated inflammation is a IBD, asthma, type 1 diabetes, multiple sclerosis, obesity, type 2 diabetes, hay fever, food allergies, skin allergies, or rheumatoid arthritis. Reference is also made to Mazmanian et al 2008⁴³, in particular the figures and related portion of the paper herein incorporated by reference in its entirety.

In some embodiments, the inflammation is a systemic inflammation. Systemic inflammations include but are not limited to an inflammatory response in the circulatory system, an inflammatory response which is not confined in a specific organ, and an inflammatory response that extends to a plurality (up to all) tissues and organs in an individual.

In some embodiments, a ZP can be used to control an inflammation associated with an imbalance of T-helper cell profile and in particular to a Th17 cell profile, including but not limited to rheumatoid arthritis, respiratory diseases, allograft rejection, systemic lupus erythematosis, tumorgenesis, multiple sclerosis, systemic sclerosis and chronic inflammatory bowel disease.

In some embodiments, PSA can be administered systemically to the individual. The wording “systemic administration” as used herein indicates a route of administration by which PSA is brought in contact with the body of the individual, so that the desired effect is systemic (i.e. non limited to the specific tissue where the inflammation occurs). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In some embodiments, administration is performed intravenously by introducing a liquid formulation including a ZP in a vein of an individual using intravenous access methods identifiable by a skilled person, including access through the skin into a peripheral vein. In some embodiments, administration of a ZP is performed intraperitoneally, by injecting a ZP in the peritoneum of an individual, and in particular of animals or humans. Intraperitoneal administration is generally preferred when large amounts of blood replacement fluids are needed, or when low blood pressure or other problems prevent the use of a suitable blood vessel for intravenous injection. In some embodiments administration is performed intragastrically, including administration through a feeding tube. In some embodiments, administration of a ZP is performed intracranially. In some embodiments a ZP can be administered topically by applying the ZP usually included in an appropriate formulation directly where its action is desired. Topical administration include but is not limited to epicutaneous administration, inhalational administration (e.g. in asthma medications), enema, eye drops (E.G. onto the conjunctiva), ear drops, intranasal route (e.g. decongestant nasal sprays), and vaginal administration.

In some embodiments, the inflammation is an inflammation of in a tissue and in particular in pancreas, lungs, joints, skin, brains and central nervous system, and eyes.

In some embodiments, PSA is used in a method of treating or preventing a condition associated with inflammation in an individual. The method comprises administering to the individual a therapeutically effective amount of the PSA. The term “individual” as used herein includes a single biological organism wherein inflammation can occur including but not limited to animals and in particular higher animals and in particular vertebrates such as mammals and in particular human beings.

The term “condition” as used herein indicates a usually the physical status of the body of an individual, as a whole or of one or more of its parts, that does not conform to a physical status of the individual, as a whole or of one or more of its parts, that is associated with a state of complete physical, mental and possibly social well-being. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms. Exemplary conditions include but are not limited to injuries, disabilities, disorders (including mental and physical disorders), syndromes, infections, deviant behaviors of the individual and atypical variations of structure and functions of the body of an individual or parts thereof.

The wording “associated to” as used herein with reference to two items indicates a relation between the two items such that the occurrence of a first item is accompanied by the occurrence of the second item, which includes but is not limited to a cause-effect relation and sign/symptoms-disease relation.

Conditions associated with an inflammation include but are not limited to inflammatory bowel disease, including but not limited to Chron's disease and ulcerative colitis, asthma, dermatitis, arthritis, myasthenia gravis, Grave's disease, sclerosis, psoriasis.

The term “treatment” as used herein indicates any activity that is part of a medical care for or deals with a condition medically or surgically.

The term “prevention” as used herein indicates any activity, which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

An effective amount and in particular a therapeutically effective amount of PSA is for example in the range of between about 1 μg to about 100 μg of PSA per 0.025 kilograms of body weight. In some embodiments, the effective amount is in a range from about 001 to about 1,000 μg per 25 grams of body weight.

In some embodiments, PSA is comprised in a composition together with a suitable vehicle. The term “vehicle” as used herein indicates any of various media acting usually as solvents, carriers, binders or diluents for PSA comprised in the composition as an active ingredient.

In some embodiments, where the composition is to be administered to an individual the composition can be a pharmaceutical anti-inflammatory composition, and comprises PSA and a pharmaceutically acceptable vehicle.

In some embodiments, PSA can be included in pharmaceutical compositions together with an excipient or diluent. In particular, in some embodiments, pharmaceutical compositions are disclosed which contain PSA, in combination with one or more compatible and pharmaceutically acceptable vehicle, and in particular with pharmaceutically acceptable diluents or excipients.

The term “excipient” as used herein indicates an inactive substance used as a carrier for the active ingredients of a medication. Suitable excipients for the pharmaceutical compositions herein disclosed include any substance that enhances the ability of the body of an individual to absorb PSA. Suitable excipients also include any substance that can be used to bulk up formulations with PSA to allow for convenient and accurate dosage. In addition to their use in the single-dosage quantity, excipients can be used in the manufacturing process to aid in the handling of PSA. Depending on the route of administration, and form of medication, different excipients may be used. Exemplary excipients include but are not limited to antiadherents, binders, coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluent include any substance that can decrease the viscosity of a medicinal preparation.

In certain embodiments, compositions and, in particular, pharmaceutical compositions can be formulated for systemic administration, which includes enteral and parenteral administration.

Exemplary compositions for parenteral administration include but are not limited to sterile aqueous solutions, injectable solutions or suspensions including PSA. In some embodiments, a composition for parenteral administration can be prepared at the time of use by dissolving a powdered composition, previously prepared in lyophilized form, in a biologically compatible aqueous liquid (distilled water, physiological solution or other aqueous solution).

Exemplary compositions for enteral administration include but are not limited to a tablet, a capsule, drops, and suppositories.

The Examples section of the present disclosure illustrates examples of the compositions and methods herein described as well as the studies carried out by applicants in order to investigate the functional and physical interactions of PSA.

Further advantages and characteristics of the present disclosure will become more apparent hereinafter from the following detailed disclosure in the Examples given by way or illustration only with reference to an experimental section.

### EXAMPLES

The methods and system herein disclosed are further illustrated in the following examples, which are provided by way of illustration and are not intended to be limiting.

In particular, in the following examples, the following materials and methods were used.

Bacterial Strains and Animals.

B. fragilis NCTC9343 and H. hepaticus ATCC51149 were obtained from the American Type Culture Collection. Conventionally reared SPF mice of strains C57BL/6NTac, C57BL/6NTac IL-10^{-/-}, and B6.129S6-Rag2^{tmlFwa} N12 (Rag2^{-/-}) were purchased from Taconic Farms (Germantown, N.Y.) and screened negative for B. fragilis and H. hepaticus. Swiss-Webster germ-free (SWGF) mice were purchased from Taconic Farms. Upon delivery in sterile shipping containers, the mice were transferred to sterile isolators (Class Biologically Clean, Madison, Wis.) in our animal facility. Animals were screened weekly for bacterial, viral, and fungal contamination as previously described⁴⁰. All animals were cared for under established protocols and the IACUC guidelines of Harvard Medical School and the California Institute of Technology.

Model of Inflammation:

Three models of intestinal inflammation were used: 1) CD4⁺CD45Rb^{high} T cells were purified from the spleens of wild-type or IL-10^{-/-} donor mice by flow cytometry and transferred into Rag^{-/-} (C57B1/6) recipients as described. 2) TNBS colitis was induced by pre-sensitization of wild-type (C57B1/6) mice on the skin with a TNBS/acetone mix. Seven days after sensitization, 2.5% TNBS in ethanol was administered rectally; mice were sacrificed 3-6 days later. 3) IL10^{-/-} mice were colonized (by oral gavage) with H. hepaticus alone or in combination with wild-type B. fragilis or B. fragilis APSA.

Assays and Scoring Systems:

Cytokines from the spleen, colons, or MLNs were assayed by ELISA, Q-PCR, or flow cytometry. Colitis was assessed with tissue sections (fixed, paraffin embedded, sectioned onto a slide, and stained with hematoxylin and eosin) and was scored by a blinded pathologist (Dr. R. T. Bronson, Harvard Medical School) according to a standard scoring system: 0, no thickening of colonic tissues and no inflammation (infiltration of lymphocytes); 1, mild thickening of tissues but no inflammation; 2, mild thickening of tissues and mild inflammation; 3, severe thickening and severe inflammation. BMDCs were purified from femurs of mice after extraction and washing in PBS. Cells were cultured for 8 days in C-RPMI-10 in the presence of GM-CSF (20 ng/mL; Biosource, Camarillo, Calif.). CD4⁺ T cells were purified by negative selection over a magnetic column (Miltenyi or R& D Systems).

Flow Cytometry, Fluorescence-Activated Cell Sorting (FACS), and Staining.

Lymphocytes were isolated from mouse spleens that were mechanically disrupted into single-cell preparations. Red blood cells were lysed, and splenocytes (1×10⁶) were incubated with various combinations of antibodies (BD Pharmingen, San Diego, Calif.) at 2 mg/mL for 30 min at 4° C. Cells were then washed and either fixed or used directly. For intracellular cytokine flow cytometry, samples were analyzed on a model FC500 cytometer (Beckman Coulter, Fullerton, Calif.) or a FacsCalibur (Becton Dickson), and data were analyzed with RXP Analysis Software (Beckman Coulter) or FlowJO. FACS was performed on a BD FACSAria, and cell purity was always >99%.

In Vitro Cytokine Assays.

For colon organ cultures, procedures were followed as previously reported⁴¹. For co-culture, CD4⁺ T cells were purified from splenic lymphocytes (prepared as described above) with a CD4⁺ T Cell Subset Kit (R&D Systems, Minneapolis, Minn.) used as instructed by the manufacturer. Cell purity was always >95%. BMDCs were purified from femurs of mice after extraction and washing in PBS. Cells were cultured for 8 days in C-RPMI-10 in the presence of GM-CSF (20 ng/mL; Biosource, Camarillo, Calif.). Medium was replaced after 4 days, and adherent cells were cultured for an additional 4 days, at which point nonadherent cells were recovered, washed, and used directly. Cells were >95% CD11c⁺ at the time of use. Purified CD4⁺ T cells (1×10⁶) were mixed with purified CD11c⁺ BMDCs (1×10⁶) in a 48-well plate and were incubated at 37° C. in an atmosphere containing 5% CO₂. Various stimuli were used, as described in Results. ELISA was performed with pre-coated plate kits (BD Pharmingen) according to the manufacturer's guidelines. In some assays, H. hepaticus, with or without wild-type B. fragilis or B. fragilis APSA, was added at various concentrations.

Induction of Experimental Colitis.

As assessed by PCR, Rag2^{-/-} and control C57B1/6 mice were negative for H. hepaticus colonization at the time of delivery. Splenic lymphocytes were harvested from wild-type donor mice, and CD4⁺CD45Rb^{high} cells were purified from lymphocyte populations by FACS as described above. Cells were washed with PBS, and 3×10⁵ cells were injected intraperitoneally in a volume of 0.2 mL into recipient H. hepaticus-colonized Rag2^{-/-} animals. For colonization experiments, both H. hepaticus (1×10⁸ organisms) and B. fragilis (1×10⁸ organisms) were introduced at the time of cell transfer. Throughout PSA treatment studies, animals received 50 μg of PSA by gavage 3 times per week. Animals were weighed throughout the experiment until sacrifice at 8 weeks.

Induction of Intestinal Inflammation-TNBS Colitis.

The backs of wild-type (C57BL/6) male mice were shaved, and pre-sensitization solution (150 μL; acetone with olive oil in a 4:1 ratio mixed with 5% TNBS in a 4:1 ratio) was slowly applied. Seven days after sensitization, mice were anesthetized with isofluorene and TNBS solution (100 μL; 1:1 5% TNBS with absolute ethanol) administered rectally through a 3.5F catheter (Instech Solomon; SIL-C35). Mice were analyzed 4-6 days after TNBS administration.

Histologic Tissue Analysis.

Mouse tissues in Bouin's fixative (VWR, West Chester, Pa.) were embedded in paraffin, sectioned (6-μm slices), mounted onto slides, and stained with hematoxylin and eosin. Sections were evaluated in blinded fashion by a single pathologist (Dr. R. T. Bronson, Harvard Medical School).

Quantitative Real-Time PCR.

RNA was extracted with Trizol per the manufacturer's instructions (Invitrogen). RNA (1 μg) was reverse transcribed into cDNA with an iScript cDNA synthesis kit (Bio-Rad). cDNA was diluted by addition of 60 μL of water, and a 2-μL volume of this solution was used for Q-PCR. Q-PCR was performed using IQ SYBR Green supermix (Bio-Rad) and primers were used at 0.2 μm. Q-PCR was performed on a Bio-Rad iCycler IQ5. Sequences of Q-PCR primers were as follows 5′-3′: IL-23 (p19) F: AGC TAT GAA TCT ACT AAG AGA GGG ACA (SEQ ID NO: 5) R: GTC CTA GTA GGG AGG TGT GAA GTT G (SEQ ID NO: 6). IL-17A F: TTA AGG TTC TCT CCT CTG AA (SEQ ID NO: 7) R: TAG GGA GCT AAA TTA TCC AA. (SEQ ID NO: 8) TNFaF: ACG GCA TGG ATC TCA AAG AC (SEQ ID NO: 9) R: GTG GGT GAG GAG CAC GTA GT (SEQ ID NO: 10). IL-10 F: CTG GAC AAC ATA CTG CTA ACC G (SEQ ID NO: 11) R: GGG CAT CAC TTC TAC CAG GTA A (SEQ ID NO: 12) RORyT F: CCG CTG AGA GGG CTT CAC (SEQ ID NO: 13) R: TGC AGG AGT AGG CCA CAT TAC A (SEQ ID NO: 14) IL-21 F: ATC CTG AAC TTC TAT CAG CTC CAC (SEQ ID NO: 15) R: GCA TTT AGC TAT GTG CTT CTG TTT C (SEQ ID NO: 16) IL-27 F: CTG TTG CTG CTA CCC TTG CTT (SEQ ID NO: 17) R: CAC TCC TGG CAA TCG AGA TTC (SEQ ID NO: 18).

### Example 1

### PSA Balances the Th1/Th2 Profile of the Mammalian Immune System

The two subtypes of effector CD4⁺ T cells, T_{H}1 and T_{H}2, are defined by expression of the cytokines interferon g (IFNg) and interleukin 4 (IL-4), respectively (Janeway et al., 2001). As shown above, PSA induces CD4⁺ T cell expansion in B. fragilis-colonized mice and in vitro. To further characterize the effects of PSA-mediated T cell activation, we assessed cytokine profiles using purified cellular components. Co-culture of DCs and CD4⁺ T cells in the presence of PSA yields dose-dependent up-expression of the T_{H}1 cytokine IFNg. The level of IFNg production associated with PSA is comparable to that associated with several known potent IFNg inducers (a-CD3, LPS, and staphylococcal enterotoxin A [SEA]) and requires both DCs and T cells. Specificity is evidenced by the lack of T_{H}1 cytokine production after NAc-PSA treatment.

T_{H}1 cytokine production suppresses T_{H}2 responses; conversely, T_{H}2 cytokine expression inhibits T_{H}1 responses. Normal immune responses require a controlled balance of these opposing signals. Examination of IL-4 expression in response to PSA treatment reveals no cytokine production by purified CD4⁺ T cells. a-CD3 and the superantigen SEA are potent stimulators of both classes of cytokine. As T_{H}2 cytokine production is a “default pathway” in many systems (Kidd, 2003; Amsen et al. 2004)) and T_{H}1 cytokine production is antagonistic to T_{H}2 expression, the specific stimulation of IFNg by PSA in vitro may provide a mechanism for establishing commensal-mediated homeostasis of the host immune system by balancing T_{H}1/T_{H}2 responses.

### Example 2

### PSA Is Required for Appropriate CD4⁺ T-Helper Cytokine Production During Colonization

A proper T_{H}1/T_{H}2 balance is critical for human and animal health; over- or underproduction of either response is associated with immunologic disorders. We investigated the effects of PSA on T_{H}1/T_{H}2 cytokine responses in colonized animals, again using germ-free mice. CD4⁺ T cells from mouse spleens were purified and tested by ELISA for cytokine production. Overproduction of the T_{H}2 cytokine IL-4 in spleens of germ-free mice compared with levels in conventional mice. This result is consistent with previous reports of the appreciably T_{H}2-skewed profile of mice devoid of bacterial contamination and reflects the human neonatal (precolonization) cytokine profile (Kirjavainen and Gibson, 1999; Prescott et al., 1998; Adkins, 2000; Kidd, 2003). This “default” T_{H}2-bias in the absence of bacterial colonization again highlights the profound contributions of the microflora to immune development and provides a model to test the effects of symbiotic bacteria on the establishment of appropriate host cytokine production.

Mice colonized with wild-type B. fragilis alone display a level of IL-4 production similar to that in conventional mice with a complex microflora; this similarity shows the organism's sufficiency to correct systemic immune defects. Moreover, mice colonized with B. fragilis DPSA produce T_{H}2 cytokines at elevated levels similar to those in germ-free mice. Thus the expression of a single bacterial antigen allows B. fragilis to correct the IL-4 cytokine imbalance found in uncolonized animals.

Examination of IFNg production by purified splenic CD4⁺ T cells reveals that germ-free mice, which are T_{H}2-skewed, are deficient in production of this prototypical T_{H}1 marker when compared to conventional mice. Colonization with wild-type B. fragilis alone is sufficient to correct the defect in IFNg expression in germ-free mice, with levels nearly as high as those in conventional mice. Lack of PSA production by the B. fragilis mutant during colonization of germ-free mice results in low-level production of T_{H}1 cytokines. These results were corroborated by intracellular cytokine staining of splenic lymphocytes from each group, which confirms that IFNg production is attributable to CD4⁺ T cells. The production of IL-2, another T_{H}1 cytokine, by CD4⁺ T cells in gnotobiotic mice also requires PSA production data not shown) Together, these results demonstrate that intestinal colonization of germ-free mice by B. fragilis alone is sufficient to establish a proper systemic T_{H}1/T_{H}2 balance within the host—a fundamental aspect of the mammalian immune response required for health.

### Example 3

### PSA Suppresses Th-17 Induced Inflammation

Experimental colitis and human IBD result from an initial inflammatory response that—lacking repression—advances in an uncontrolled fashion and ultimately leads to intestinal pathology and disease. To elucidate how PSA affects these primary inflammatory responses, Applicants employed an animal model of chemically induced colonic inflammation. Rectal administration of trinitrobenzene sulphonic acid (TNBS) to wild-type mice mimics the initiation of colitis by eliciting inflammatory T cell responses. Disease was induced by administration of TNBS (or vehicle, as a negative control), and oral treatment of PSA was evaluated.

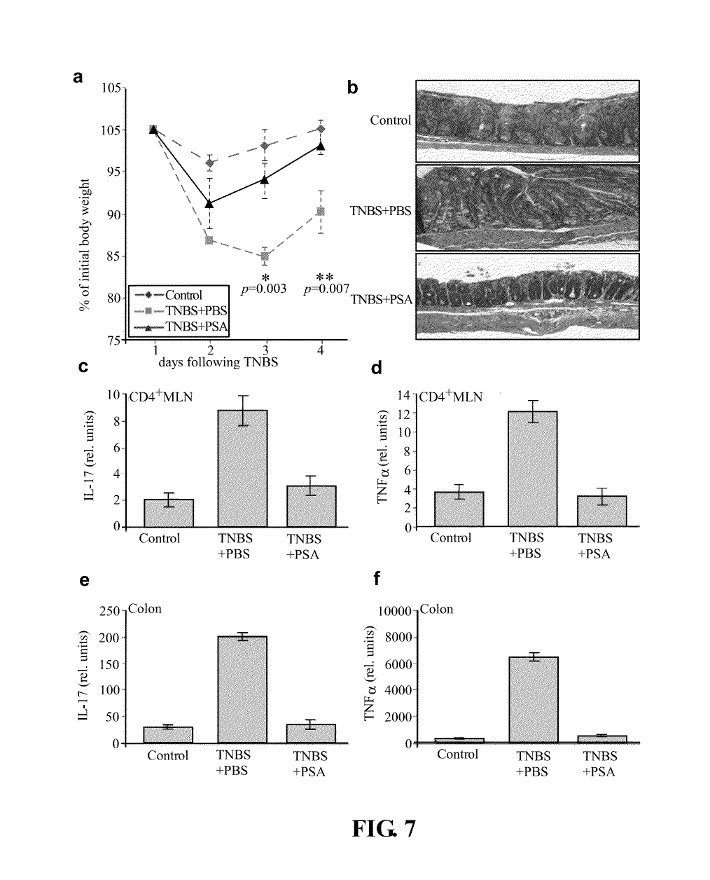

The results illustrated in FIG. 7 show that the intestinal immune response are beneficially modulated by PSA. In particular, the results illustrated in FIG. 7a show that TNBS-treated animals display weight loss that is statistically significant relative to figures for vehicle-treated and PSA-treated animals, although partial weight loss is observed in the PSA group (FIG. 7a). Histological analysis confirmed PSA protection of colonic tissues against the massive epithelial hyperplasia and loss of colonocyte organization seen after TNBS treatment (FIG. 7b). Studies have shown that pathogenic T_{H}17 cells, which produce IL-17, mediate the induction of experimental colitis³⁰. Indeed, IL-17 levels are increased among purified CD4⁺ T cells from mesenteric lymph nodes (MLNs; FIG. 7c) of diseased animals but not from those of PSA-treated animals. The increased level of TNFa among CD4⁺ T cells from MLNs of TNBS-treated animals is also reduced in PSA-treated groups (FIG. 7d). Transcriptional analysis of TNBS-treated colons revealed that expression of both IL-17 and TNFa is highly elevated in diseased but not in PSA-protected animals (FIGS. 7e and 7f).

Therefore, the above results show that PSA inhibits intestinal pathology and inflammation in a chemically induced model of experimental colitis.

### Example 4

### PSA Induces the Differentiation of IL-10 Producing Treg to Suppress Inflammation

Protection from experimental colitis is engendered through anti-inflammatory processes that prevent undesirable reactions against the intestinal microbiota²³. Interleukin-10-deficient (IL-10^{-/-}) animals develop colitis³¹. IL-10, one of the most potent anti-inflammatory cytokines, is required for protection in many animal models of inflammation^{21, 27, 32}.

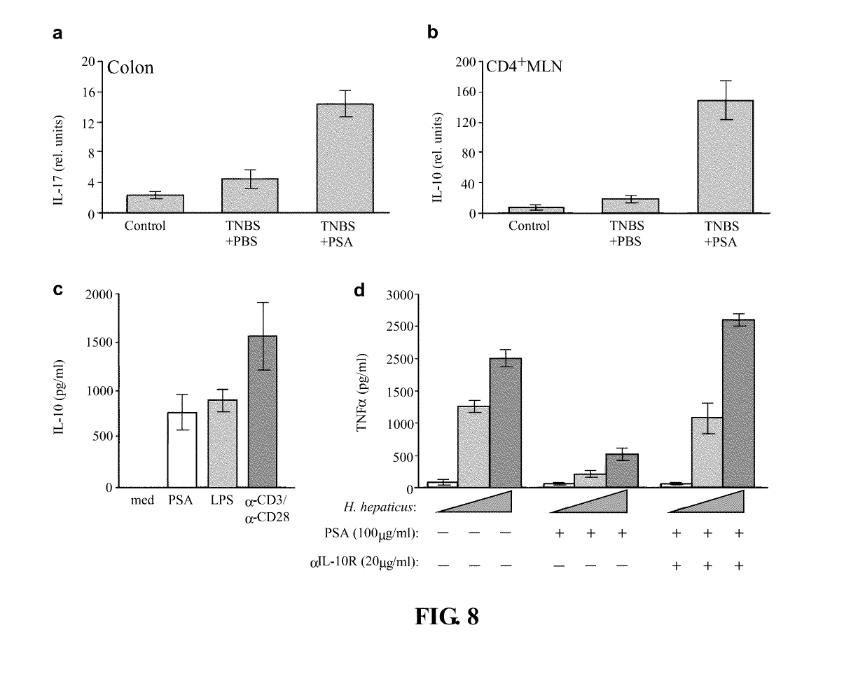

The results of a series of experiments directed to test the effect of PSA on IL-10 production are illustrated in FIG. 8, and show that PSA induces IL-10 expression in TNBS-treated animals and inhibits pro-inflammatory cytokine production in primary cultured cells through IL-10 production. In particular, as assayed by real-time PCR, transcriptional levels of IL-10 within colons of PSA-treated mice are significantly higher than those in control and TNBS-treated mice (FIG. 8a). IL-10 is produced by many cell types. However, since CD4⁺ T cells that express IL-10 display immunosuppressive activities that inhibit inflammation during experimental colitis³³, Applicants tested the IL-10 production in CD4⁺ T. When fresh CD4⁺ T cells were purified from MLNs of PSA-treated mice (in which inflammation is reduced), highly elevated levels of the IL-10 transcript were observed (FIG. 8b). Applicants then assessed whether PSA is sufficient to induce IL-10 in vitro; when bone marrow-derived dendritic cells (BMDCs) and naïve CD4⁺ T cells were treated with purified PSA, a specific increase in IL-10 production was observed (FIG. 8c).

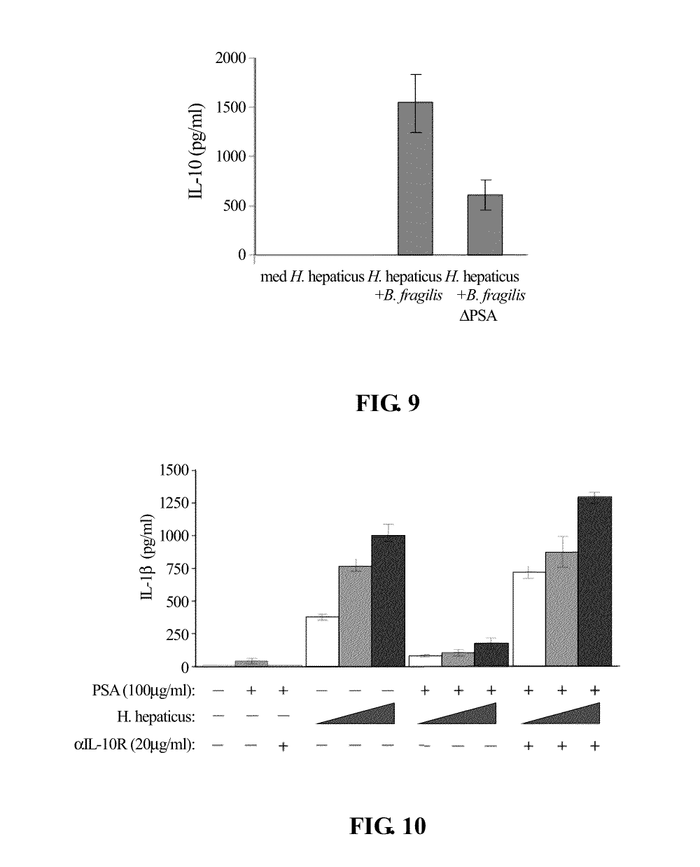

A further series of experiments illustrated in FIG. 9, shows that PSA from B. fragilis induces expression of IL-10 in vitro. In particular, BMDCs and naïve CD4⁺ T cells were infected with H. hepaticus co-cultured with B. fragilis, and a specific expression of IL-10 from culture supernatants was observed; co-culture with B. fragilis DPSA induces significantly lower levels of IL-10 (FIG. 9). Since PSA induces expression of IL-10 in vitro, to test whether this molecule is required for inhibition of inflammatory responses to H. hepaticus, BMDC-T cell co-cultures were infected with live H. hepaticus and measured expression of the critical pro-inflammatory cytokine TNFa. Addition of increasing concentrations of the pathogenic commensal causes a dose-dependent increase in TNFa production, as measured by ELISA of culture supernatants (FIG. 8d; left three bars). Treatment of cells with purified PSA markedly decreases TNFa production in response to H. hepaticus (FIG. 8d; middle three bars). Most importantly, co-incubation of cell cultures with H. hepaticus and PSA in the presence of a neutralizing IL-10 receptor antibody (aIL-10R) completely reverses this phenotypic effect and increases expression of TNFa (FIG. 8d; right three bars).

The results are similar for the related pro-inflammatory cytokine IL-1b, as shown by the results of experiments illustrated in FIG. 10. In particular, infection of BMDC-T cell co-cultures with increasing concentrations of live H. hepaticus (see FIG. 10 multiplicity of infection: 0.1, 1.0, and 10, as depicted by triangles) results in release of the cytokine IL-1b Treatment of infected cells with PSA reduces IL-1b levels, as shown in the middle three bars. Neutralization of IL-10 signaling by addition of an IL-10 receptor antibody (aIL-10R) alleviates suppression of in vitro inflammatory responses, resulting in increased levels of IL-1b FIG. 10 left three bars.

Thus, the results illustrated in the present example support the conclusion that IL-10 produced in response to PSA is required for inhibition of inflammatory reactions in cell cultures.

### Example 5

### PSA Administration Results in Differentiation of Treg, Inhibition of TNF-a and IL-17 Cytokine Production and in Colitis Suppression

Applicants investigated the requirement for IL-10 in suppression of intestinal inflammation. Initially, IL-10^{-/-} animals were colonized with H. hepaticus alone or in combination with B. fragilis (wild-type or DPSA). Applicants subsequently harvested MLNs and re-stimulated cells in culture with soluble Helicobacter antigens in an assay previously developed to measure antigen-specific responses to H. hepaticus²⁷. In particular, IL-10^{-/-} mice were left uncolonized (control) or were colonized with H. hepaticus (to induce inflammation) either alone or in combination with B. fragilis (wild-type or ΔPSA). MLNs from experimental groups were pooled and re-stimulated with soluble Helicobacter antigen (5 μg/ml) for 48 hours. Secretion of pro-inflammatory cytokines TNFα (a) and IL-17A (b) was analyzed by ELISA.

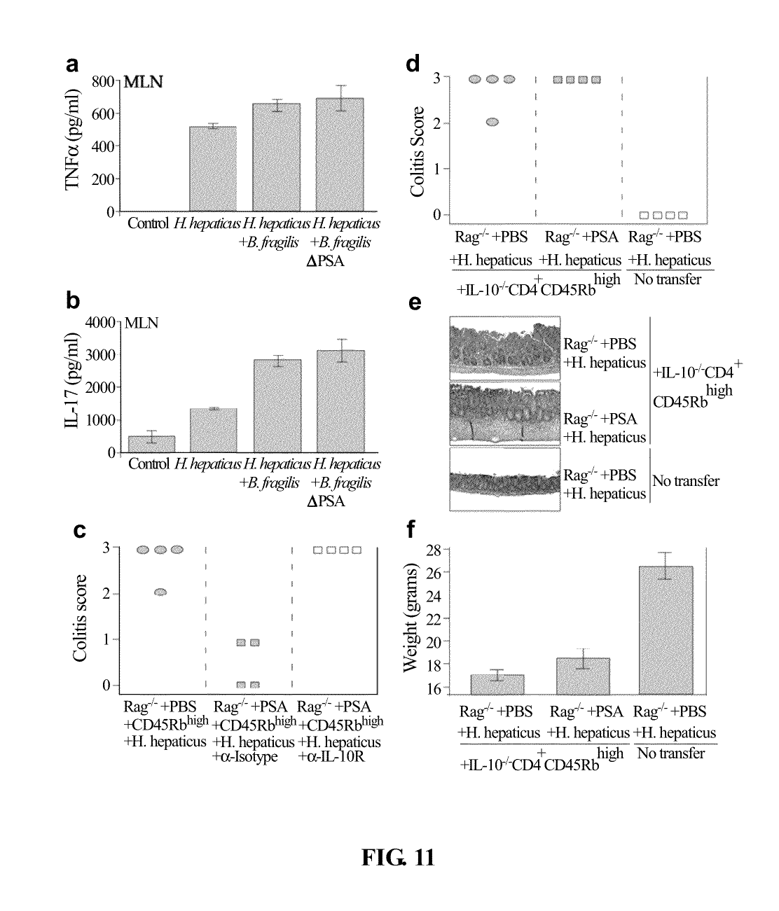

The results of these experiments, illustrated in FIG. 11a-11c, show that Helicobacter-colonized animals display increased production of TNFa and IL-17; however, in the absence of IL-10 production in colonized animals, B. fragilis co-colonization dos not reduce levels of these pro-inflammatory molecules (FIGS. 11a and b, respectively). As expected, the absence of PSA has no effect. Using the cell transfer model of colitis (see Examples 6 to 8 below, Applicants transferred CD4⁺CD45Rb^{high} T cells to Helicobacter-colonized Rag^{-/-} animals. Administration of aIL-10R to mice (to block IL-10 signaling) during oral treatment with PSA abrogates protection from colitis (FIG. 11c). In particular, colitis scores show that PSA protection requires aIL-10 signaling, as neutralizing antibodies to IL-10 block PSA's suppressive activity. Treatment with IL-10R abrogates PSA-mediated protection. (FIG. 11c).

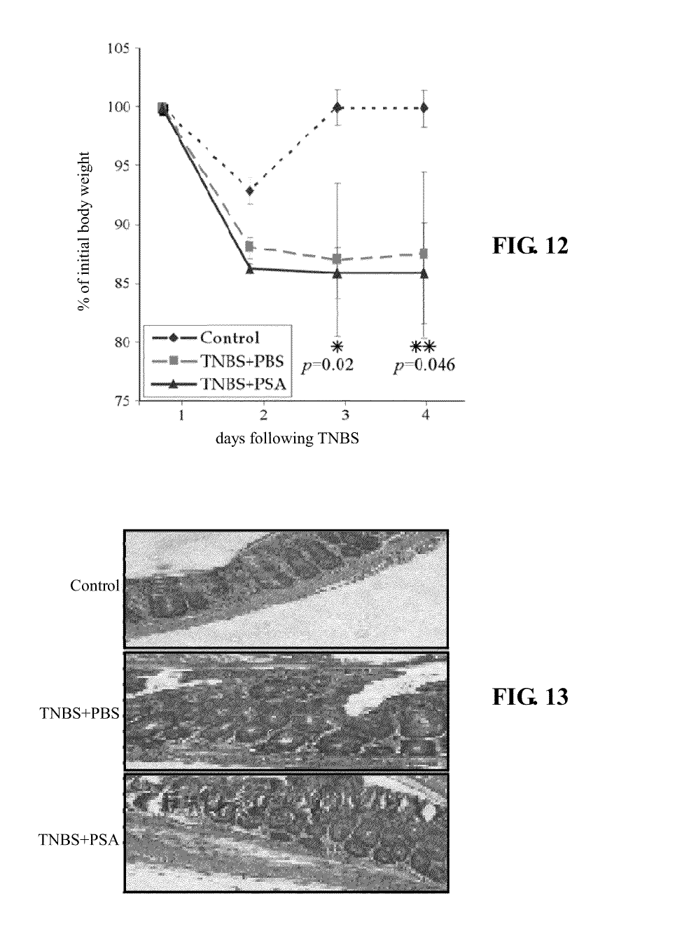

Additionally, when IL-10^{-/-} animals were treated with TNBS in the presence or absence of PSA, weight and histology data illustrated in FIGS. 12 and 13, indicated that IL-10 production is required for PSA-elicited reduction of intestinal immune responses. In particular, in a first series of experiments, groups of 4 C57BL/6 mice were treated with PSA (or PBS) and then subjected to rectal administration of TNBS or vehicle (control). SD values illustrated in FIG. 12, indicate that, in the absence of IL-10, PSA cannot restore TNBS-induced weight loss. ANOVA demonstrates that weight loss in both TNBS-treated groups is statistically different from that in control animals and that PSA does not prevent weight loss in TNBS-treated IL-10^{-/-} animals (FIG. 12).

In a second series of experiments, groups of 4 C57BL/6 mice were treated with PSA (or PBS) and then subjected to rectal administration of TNBS or vehicle (control). Histologic analysis of H&E-stained sections from a representative animal from each group is shown in FIG. 13. Thickening of the colon and epithelial hyperplasia are noted in both TNBS-treated groups of IL-10^{-/-} animals, regardless of PSA treatment. Thus, the results illustrated in FIG. 13 show that in the absence of IL-10, PSA does not reduce intestinal injury in TNBS-treated IL-10^{-/-} mice.

The above data suggest that PSA-mediated protection entails the generation and/or expansion of IL-10-producing CD4⁺ T cells. To determine whether IL-10 production by CD4⁺ T cells is required for protection, Applicants transferred CD4⁺CD45Rb^{high} T cells from IL-10^{-/-} donor mice into Rag^{-/-} recipients and then colonized the recipients with H. hepaticus.

The results illustrated in FIGS. 11d-11f show that, as expected, groups of mice receiving IL-10^{-/-} T cells along with H. hepaticus develop severe colitis (FIG. 11d; left bar) and are not protected by PSA (FIG. 11d; middle bar). This result, supported by histological findings in colons, indicates that PSA induces protection from “previously pathogenic” CD4⁺CD45Rb^{high} T cells in an IL-10-dependent manner (FIG. 11e). Weight analysis at sacrifice shows that colitic PBS- and PSA-treated animals receiving IL-10^{-/-} CD4⁺CD45Rb^{high} T cells (unlike control animals receiving no transferred cells) develop wasting disease (FIG. 11 f). Thus, IL-10 production by CD4⁺ T cells is required for PSA-mediated protection from experimental colitis. These results constitute the first reported evidence of a symbiotic bacterial molecule that networks with the immune system to coordinate anti-inflammatory responses required for mammalian health.

### Example 6

### PSA Balances the CD4⁺CD45Rb^{high}/CD4⁺CD45Rb^{low} T Cells Ratio

CD4⁺ T cells of the mammalian immune system can be generally divided into a naïve (‘uneducated’) CD4⁺CD45Rb^{high} population and an antigen-experienced (‘educated’) CD4⁺CD45Rb^{low} population¹⁶.

In a first series of experiments, mono-association of germ-free mice with wild-type B. fragilis was performed to analyze the effect on the CD4⁺CD45Rb^{low} T cells v. CD4⁺CD45Rb^{high} proportion. In particular, the ability of B. fragilis to correct deficiencies in the CD4⁺CD45Rb^{low} T cell population in spleen.

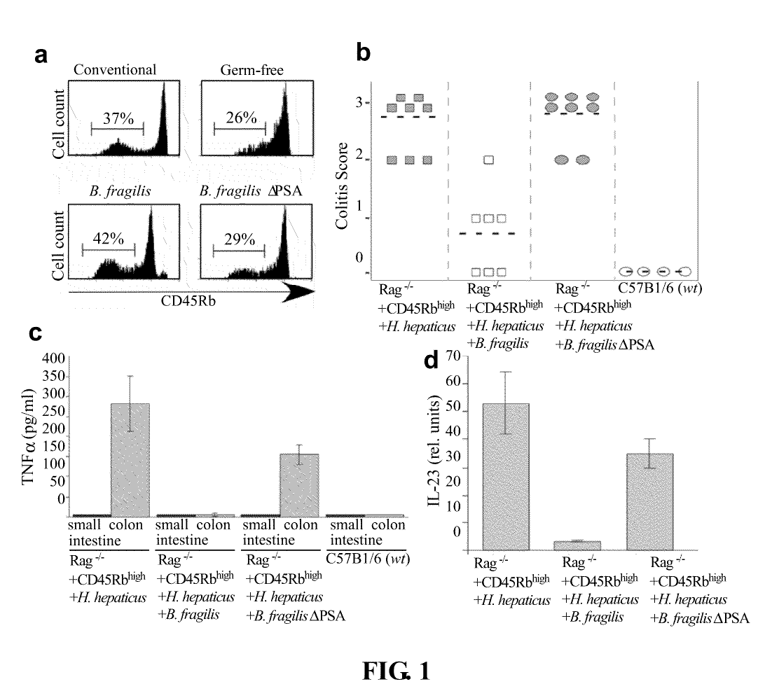

The results illustrated in FIG. 1a show that association of B. fragilis expands the proportions of CD4⁺CD45Rb^{low} T cells in a PSA-dependent manner Remarkably, Applicants found that splenic cells from germ-free animals include a smaller proportion of CD4⁺CD45Rb^{low} T cells than do those from age-matched conventional mice with a complete bacterial microbiota (FIG. 1a). Additionally, it appears that mono-colonization of germ-free mice with wild-type B. fragilis alone restores the CD4⁺CD45Rb profile in animals with a complete bacterial microbiota (FIG. 1a; left panels). Most notably, colonization with a mutant strain defective in the ability to produce PSA (B. fragilis DPSA) does not generate an expansion of the CD4⁺CD45Rb^{low} T cell population (FIG. 1a; lower right). It is well established that the latter population possesses potent anti-inflammatory properties and confers protection in animal models of inflammation¹⁷. These results suggested that PSA mediate protection from inflammation.

### Example 7

### PSA Controls IL23, IL1b and TNF-α Production in Inflamed Tissues, Thus Controlling Th17 and Th1-Mediated Cytokine Production

The well-established CD4⁺CD45Rb transfer model of experimental colitis¹⁸ was employed to investigate whether B. fragilis colonization protects animals from inflammatory disease. In this model, pathogenic CD4⁺CD45Rb^{high} T cells are separated from protective CD4⁺CD45Rb^{low} cells and transferred into specific pathogen-free (SPF) Rag^{-/-} mice. Upon cell transfer, mice are colonized with Helicobacter hepaticus^{8,19}, a pathobiont that is a benign commensal in wild-type animals but an opportunistic pathogen causing colitis in immuncompromised mice. After 8 weeks, animals are sacrificed and colitis is assessed with a standard scoring system²⁰.

The pathology scores illustrated in FIG. 1b, show that H. hepaticus colonization and CD4⁺CD45Rb^{high} T cell transfer are sufficient to induce severe colitis in Rag^{-/-} mice (FIG. 1b; first column), as previously reported^{19, 21}. Co-colonization with wild-type B. fragilis results in significant protection from disease (FIG. 1b; second column), whereas co-colonization with B. fragilis DPSA does not (FIG. 1b; third column).

Tissue damage in colitis is widely believed to result from production of inflammatory cytokines in response to commensal bacteria²². The pro-inflammatory cytokines tumor necrosis factor a (TNFa, interleukin-1b (IL-1b and IL-23 are central to disease initiation and progression in this experimental model of colitis²³. Furthermore, levels of these cytokines are elevated in patients with IBD²⁴, and therapies neutralizing TNFa have yielded promising results in clinical trials in patients with Crohn's disease²⁵. Accordingly, Applicants decided to test the inflammatory cytokine levels during disease by directly culturing intestinal tissues of T cell recipient colonized animals²⁶. The results illustrated in FIGS. 1c, 1d, 2 and 3 show that PSA alters cytokine levels in affected tissue.

In particular, the results of ELISA experiments of colon organ cultures illustrated in FIG. 1c show an increased expression of pro-inflammatory cytokine TNFa in diseased colons, with significant reductions in animals co-colonized with wild-type B. fragilis but not with B. fragilis DPSA.

The results of Q-PCR for IL-23p19 performed on splenocytes, normalized to L32 expression illustrated in FIG. 1d show that increases in IL-23 production by splenocytes following disease induction are completely suppressed by intestinal colonization with PSA-producing B. fragilis.

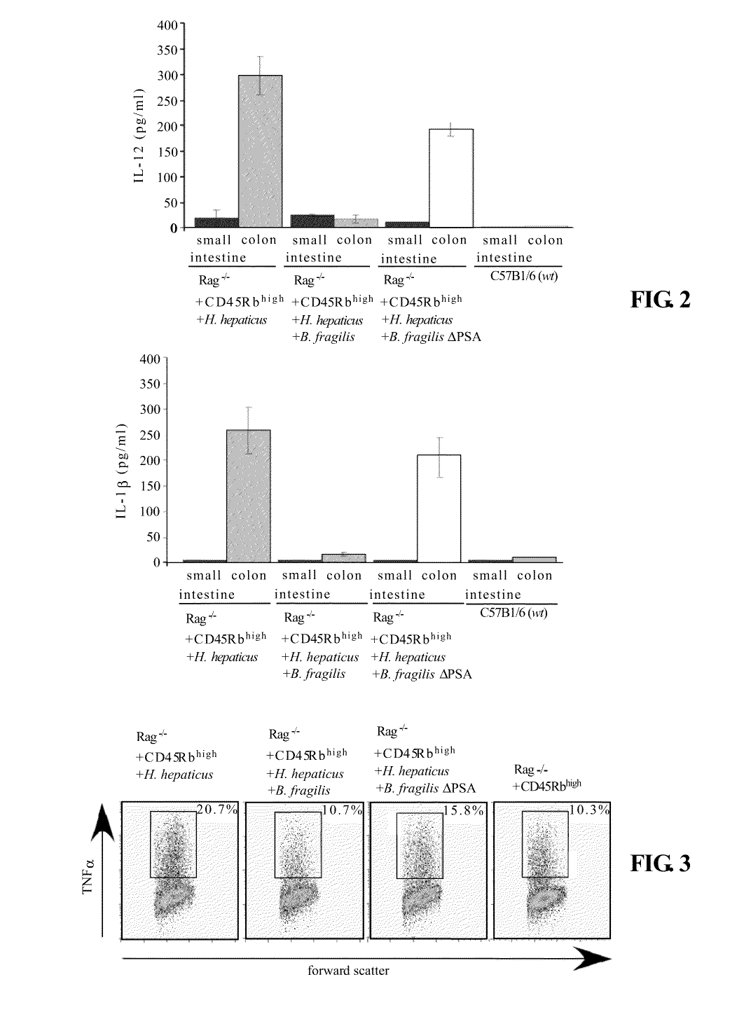

ELISA results for the pro-inflammatory cytokines IL-12p40 and IL-1b in colon and small intestines shown in FIG. 2 show a specific increase in pro-inflammatory cytokines in diseased colons but not in small intestines. This increase is significantly reduced in animals co-colonized with PSA-producing B. fragilis. Conversely, animals colonized with B. fragilis DPSA express greatly increased pro-inflammatory cytokine levels over those in control animals (C57BL/6) (FIG. 2).

The results of experiments illustrated in FIG. 3 show that the expression of the TNFa by CD4⁺ T cells is reduced by wild-type B. fragilis colonization during experimental colitis. CD4⁺ cells were purified from pooled splenocytes from each group (4 mice per group) and restimulated in vitro with PMA and ionomycin in the presence of brefeldin A for 4 hours. Cells were stained for intracellular TNFα. Cells within the lymphocyte gate were included in the analysis, and numbers indicate the percentage of cells producing TNFα. Purified cells were >90% CD4⁺. Animals colonized with PSA-producing B. fragilis during protection displayed lower TNFa levels than diseased animals.

Overall these above results show that PSA performs its effect by altering cytokine levels in affected tissues. In particular, levels of the pro-inflammatory cytokines TNFa (FIG. 1c), IL-12p40, and IL-1b (FIG. 2) are elevated in the colons of Rag^{-/-} recipient mice colonized with H. hepaticus but not in sections of small intestine (a site not affected in this model). Consistent with the protection observed by pathophysiologic analysis of experimental colitis, TNFa levels are not elevated when these animals are co-colonized with wild-type B. fragilis. T cell transfer plus co-colonization with H. hepaticus and B. fragilis DPSA results in increased colonic cytokine production similar to that seen in Rag^{-/-} animals colonized with H. hepaticus alone. Moreover, purified splenic CD4⁺ T cells from H. hepaticus-colonized animals, display increased TNFa production; this condition is corrected by colonization with wild-type B. fragilis but not with the PSA deletion strain (FIG. 3). Expression of IL-23 is critical in the cascade of events leading to experimental colitis^{27,28}. Applicants found that increases in IL-23 production by splenocytes following disease induction are completely suppressed by intestinal colonization with PSA-producing B. fragilis (FIG. 1d).

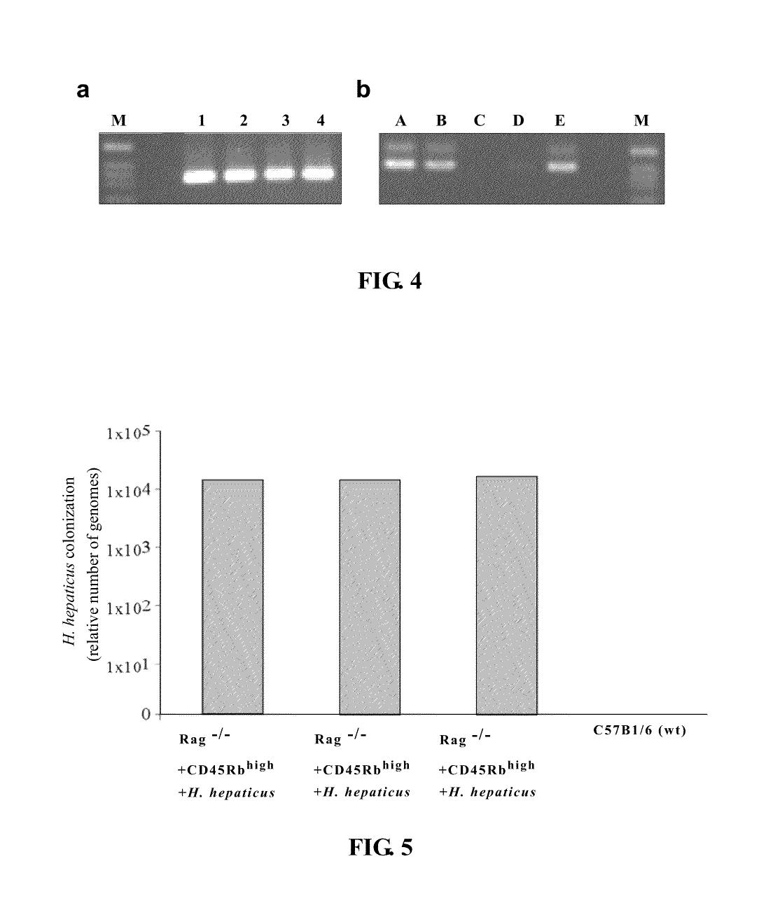

Experiments directed to rule out bacterial clearance were performed to show whether, over the course of the experiments, levels of H. hepaticus and B. fragilis colonization did differ between groups. The results illustrated in FIGS. 4 and 5 show that protection is not the result of bacterial clearance.

In particular, the results shown in FIG. 4 show that experimental animals remain colonized with H. hepaticus and B. fragilis throughout the course of disease. More particularly, the ethidium bromide-stained gel electrophoresis of H. hepaticus-specific Q-PCR of FIG. 4a shows that co-colonization with B. fragilis does not induce clearance of bacteria after 8 weeks. The primers used for H. hepaticus 16S rDNA were: (HB-15) 5′-GAAACTGTTACTCTG-3′ (SEQ ID NO: 1) and (HB-17) 5′-TCAAGCTCCCCGAAGGG-3′ (SEQ ID NO: 2). Ethidium bromide-stained gel electrophoresis of B. fragilis-specific Q-PCR of FIG. 4b show stable bacterial colonization after 8 weeks; the primers used for B. fragilis ssr3 (finB) gene were: (ssr3-F) 5′-TATTTGCGAGAAGGTGAT-3′ (SEQ ID NO: 3) and (ssr3-r) 5′-TAAACGCTTTGCTGCTAT-3′ (SEQ ID NO: 4).

In an additional series of experiments, quantitation of H. hepaticus was performed to verify whether PSA administration affected the presence of the organism. The results of quantitation of H. hepaticus colonization experiments of FIG. 5 demonstrate that the organism is present in equal numbers regardless of PSA-mediated protection. In particular, in the experiments of FIG. 5 fecal samples were collected from each experimental group, and total DNA was extracted (Qiagen DNAeasy tissue kit). Equal amounts of DNA (50 ng) were used in Q-PCR (Bio-rad) with H. hepaticus-specific primers. Q-PCR for H. hepaticus colonization was assessed according to Young et al., 2004¹ as log¹⁰ number of copies of a known gene (cytolethal distending toxin). Animals contained equivalent levels of H. hepaticus at the end of the experiment.

The results illustrated in this example support the conclusion that PSA is a specific immunomodulatory molecule that orchestrates beneficial immune responses to prevent B. Fragilis host from developing experimental colitis.

### Example 8

### PSA Suppresses Inflammation Associated with CD4⁺CD45Rb^{high} T Cells

To determine whether PSA is sufficient for protection in the absence of the intact B. fragilis organism, Applicants purified PSA to homogeneity²⁹ and administered it by gavage (orally) to Rag^{-/-} mice. Disease progression was then measured by various pathologic and histologic criteria.

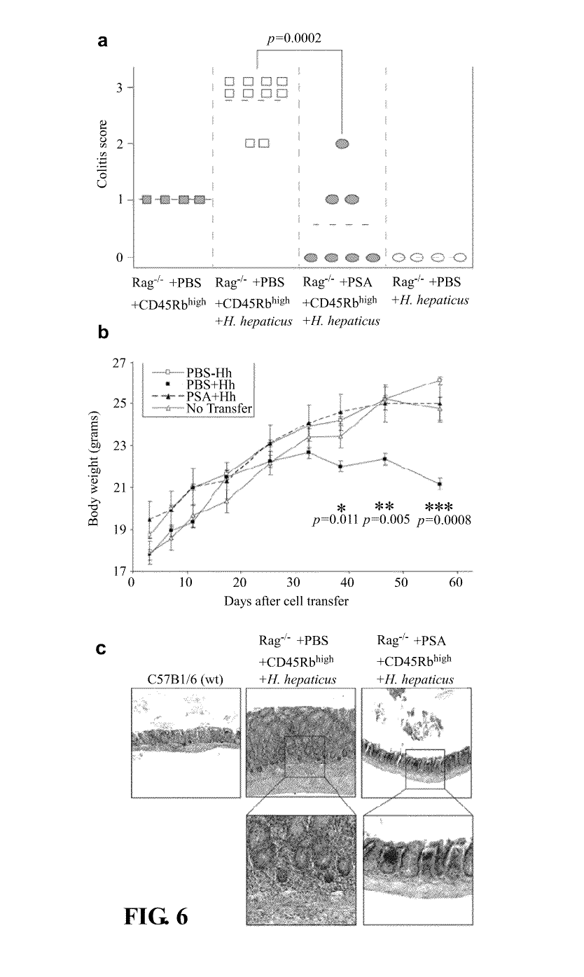

The results of related experiments illustrated in FIG. 6, show that purified PSA orally administered protects against experimental colitis.

In particular, in a first series of experiments illustrated in FIG. 6a, colitis scores after CD4⁺CD45Rb^{high} T cell transfer in the absence of H. hepaticus colonization indicated the development of very mild colitis due to inflammation elicited by the animals' SPF microbiota (FIG. 6a; first column). However, Helicobacter-colonized Rag^{-/-} animals that receive CD4⁺CD45Rb^{high} T cell transfers develop severe colitis (FIG. 6a; second column). Oral PSA administration almost completely protects animals against H. hepaticus-induced colitis (FIG. 6a; third column), reducing disease to levels of control animals without T cell transfer, that known not to develop colitis (FIG. 6a; fourth column).

A second set of experiments was then performed to test the inability to gain weight, a hallmark of colitis in this experimental setting⁴. In particular, transfer of CD4⁺CD45Rb^{high} T cells and colonization with H. hepaticus (PBS+Hh) in Rag2^{-/-} animals was performed and the animals were subsequently tested for wasting disease. The results illustrated in FIG. 6b show that wasting disease in Rag^{-/-} animals follows transfer of CD4⁺CD45Rb^{high} cells and colonization with H. hepaticus (FIG. 6b; PBS+Hh). These animals also develop intestinal pathology and express pro-inflammatory cytokines (as described above). Oral administration of PSA from the outset completely protects animals against H. hepaticus-mediated wasting disease (PSA+Hh). H. hepaticus provides the necessary antigens for inflammation induction; no pathology is observed in uncolonized animals (PBS-Hh) or in animals without cell transfer. Therefore these experiments show that oral administration of PSA protects animals against wasting (PSA+Hh).

In a further set of experiments, histologic sections of colons of wild-type animals and animals subjected to transfer of CD4⁺CD45Rb^{high} T cells and colonization with H. hepaticus (PBS+Hh) were examined to verify the presence of inflammation resulting in experimental colitis. The results illustrated in FIG. 6c show that transfer of CD4⁺CD45Rb^{high} T cells into Helicobacter-colonized Rag^{-/-} mice results in onset of severe colitis, as evidenced by massive epithelial cell hyperplasia and gross thickening of the gut wall (FIG. 6c; second panel). Furthermore, consistent with previous studies, the combination of CD4⁺CD45Rb^{high} T cell transfer plus H. hepaticus colonization results in infiltration of affected tissues by leukocytes—a hallmark of inflammation and disease (FIG. 6c second panel, bottom)^{19, 21}. Additionally, oral administration of PSA to H. hepaticus-colonized cell transfer recipients confers complete protection against experimentally induced colonic hyperplasia (FIG. 6c; third panel); furthermore, PSA-treated animals display no leukocyte infiltration in colonic tissues (FIG. 6c third panel, bottom)—a result indicating protection against inflammation.

Taken together, these results indicate that oral administration of PSA prevents colitis and protects mice against the associated weight loss and inflammatory cell infiltration observed in diseased animals.

### Example 9

### PSA is Effective in Systemic Immune Compartments Suppressing Cytokine Production by Th1 and Th17 Cells

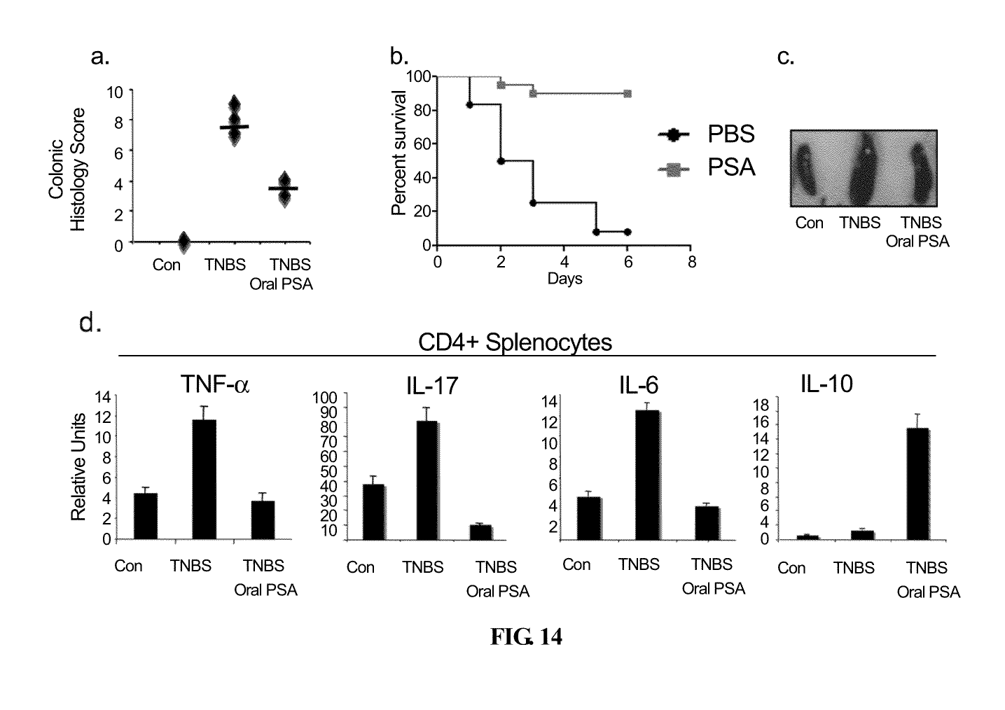

In further series of experiments, mice were treated with TNBS or TNB/PSA, orally administered to the mice. The relevant colonic sections were subsequently analyzed by a blinded pathologist who provided a histological score. The results illustrated in FIG. 14a provide further evidence that oral PSA administration reduces colitis.

While oral treatment with purified PSA protects from experimental colitis (FIG. 14a), colonization by a B. fragilis mutant that does not make PSA (B. fragilisΔPSA) is unable to protect. During the course of the experiments exemplified in Examples 1 to 8, Applicants noted strong effects of PSA in systemic immune compartments. To further understand these systemic responses Applicants utilized the TNBS induced model in the susceptible Balb/c mouse strain. As this model allows for disease induction in an immune-competent animal, it permits analysis of all immune cells involved in both disease induction and protection.

Balb/c mice were orally administered purified PSA before induction of colitis. Indeed, oral treatment of PSA protected from weight loss associated with experimental colitis and inflammation within the intestine (not shown).

Additionally, pre-treatment of Balb/c mice undergoing TNBS induced colitis, with PSA dramatically increases the survival of animals with disease from 40% to 90%, (see FIG. 14b), further attesting to the powerful anti-inflammatory effects of PSA. Since splenomegaly is commonly seen in this model of IBD and demonstrates the systemic nature of this disease, the Applicants analyzed the spleen of mice treated with TNBS, and TNBS/PSA. The results illustrated in FIG. 14c show that oral administration of ZPS is protects from the splenomegaly. Furthermore, analysis of cytokine expression showed that animals undergoing TNBS induced colitis have severe splenomegaly with increases in the expression of inflammatory cytokines from CD4+T lymphocytes residing within the spleen, as illustrated in FIG. 14d. Orally administered PSA significantly reduces splenomegaly and the expression of TNF-α, IL-17 and IL-21 in CD4+T lymphocytes from the spleen during mucosal disease (FIG. 14d). The experiments outlined in Example 5 demonstrate that PSA is able to protect from colitis through induction of IL-10 from CD4+T cells residing within the intestinal compartments. Consistent with previous data, Applicants find that IL-10 levels are elevated within the CD4+T lymphocytes in spleen (FIG. 14d). Taken together, these data suggest that PSA residing within the intestine is capable of effecting systemic immunity. In particular these results show that oral administration of ZPS can not only protect from intestinal disease but also suppresses inflammation within extra-intestinal immune compartments, such as the spleen.

### Example 10

### Parenteral Administration of PSA Protects from Inflammation and Controls TNF-α, IL-17 and IL23 Production in Intestine and Spleen

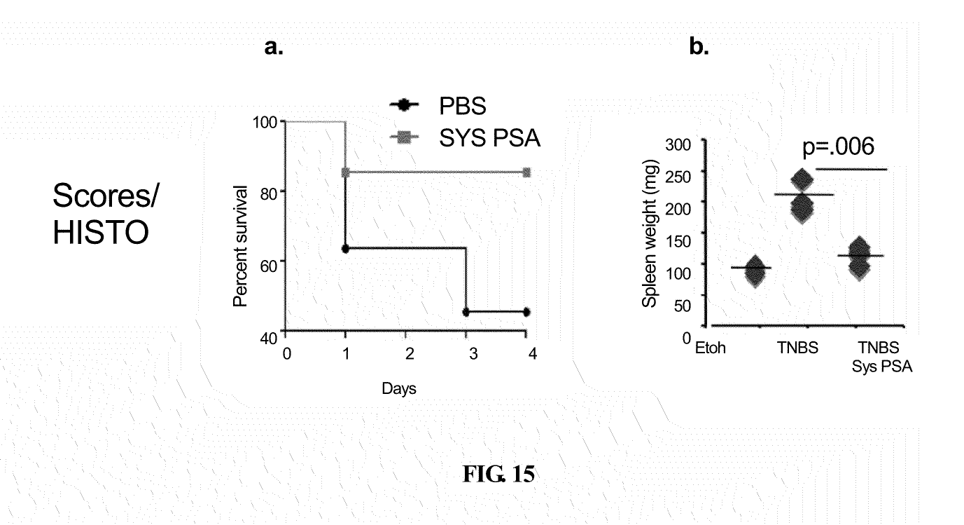

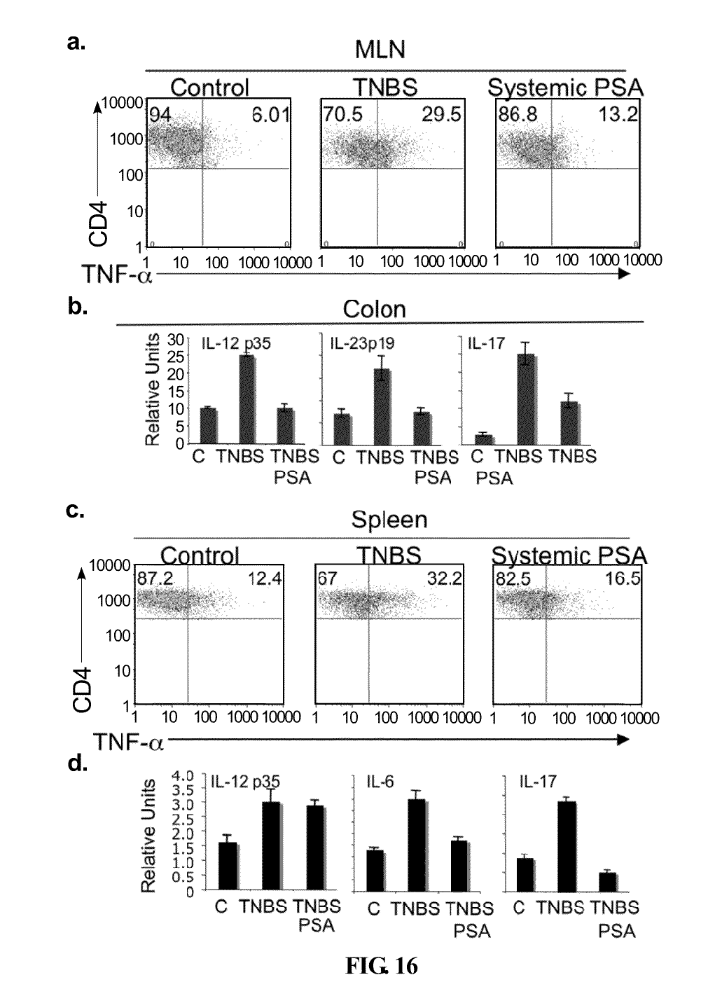

Distinct subsets of cells reside within the intestinal compartment, including CD8αα T cells, mucosal γδ T cells and CD103+ dendritic cells. Recent studies have demonstrated that these various cell types have distinct functions from their systemic immune counterparts. To determine whether PSA acts specifically within the intestine, purified PSA was administered intravenously and mucosal inflammation was induced. In a first series of experiments illustrated in FIG. 15, PSA was administered before inflammation was induced. In a second series of experiments, illustrated in FIG. 16, PSA was administered after inflammation was induced.

The results illustrated in FIG. 15, show that delivery of ZPS to extra-intestinal sites is able to protect from induced intestinal colitis. In particular, systemic administration of PSA enhances the survival of diseased animals and protects from splenomegaly (60% survival vs. 90%) (FIGS. 15a and 15b). Additionally, it is expected that colons of animals that treated with PSA systemically, have significantly less hyperplasia and inflammatory infiltrate.

The results illustrated in FIG. 16 show that while disease is exacerbated by the increased production of inflammatory cytokines at the site of induction, systemic administration of PSA during TNBS induced colitis suppresses inflammatory cytokines at both intestinal and systemic immune compartments. In particular, TNF-α from Mesenteric Lymph Nodes (MLN) CD4+T cells is increased in expression during TNBS induced colitis, but is reduced by PSA systemically administered (FIG. 16a). Additionally, inflammatory cytokines IL12p35 IL-23p19 and IL-17 are elevated in the colons of diseased mice, as shown by analysis of transcripts from RNA extracted from colons of mice undergoing TNBS induced colitis, but are reduced by administration of PSA (FIG. 16b). Also in spleen, systemic administration of ZPS reduces the production of TNF-α from CD4+T lymphocytes within the spleen as shown by the results illustrated in FIG. 16c. Furthermore, systemic administration of ZPS reduces expression of the transcripts IL-17, and IL-6 within the spleen as shown by the results illustrated in FIG. 16d.

Additional experiments also demonstrated that while PSA decreases expression of inflammatory cytokines, intravenous treatment with PSA leads to an elevation in the production of IL-10 within the intestine (supplementary data). These data indicate that systemically administered PSA is capable of extending to mucosal sites and protecting from inflammatory bowel disease.

The data illustrated in this example also show that systemic administration of PSA during TNBS induced colitis suppresses inflammatory cytokines at both intestinal and systemic immune compartments.

### Example 11

### Parenteral Administration of PSA Modulates Cytokine Expression and Protects from Systemic Inflammation Caused by Th1 and Th17 Cells

Endotoxic shock occurs during severe gram-negative bacterial infections and is characterized by hypotension, multi-organ failure and potentially death. This syndrome results from the production of multiple inflammatory cytokines, including TNF-a and IL-6, in response to the lipopolysaccharides (LPS) found in the cell wall of gram negative bacteria. IL-10 has been demonstrated to be a central regulator of the inflammatory response to LPS, indeed a single dose of IL-10 prevents death in murine models of endotoxic shock⁴². The dramatic effects of PSA within the systemic immune compartments lead us to investigate whether PSA could ameliorate systemic inflammation.

To determine whether PSA was capable of suppressing inflammation associated with endotoxic shock Applicants injected Balb/c mice with a low dose (100 ug) of LPS and monitored serum levels of the cytokines TNF-α and IL-6. In particular, serum was collected from mice 1 and 4 hours post-administration of 100 μg or 500 μg of LPS and TNF-α and IL-6 protein levels in the serum were determined by ELISA.

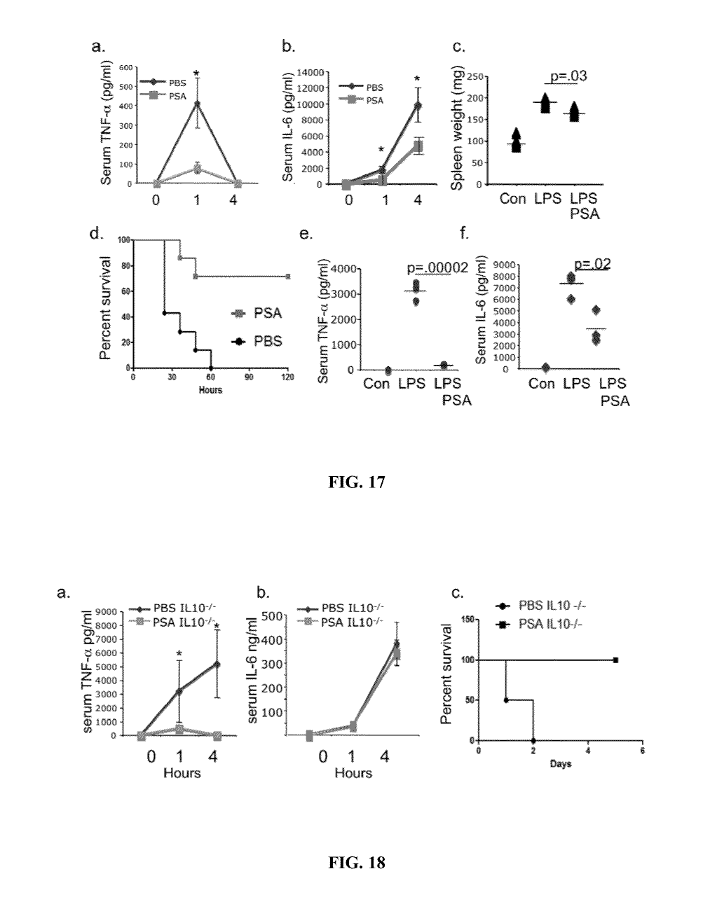

The results illustrated in FIG. 17a to c, show that untreated mice had undetectable levels of serum TNF-α and IL-6 at both time points collected. In particular, consistent with previous studies, in absence of PSA administration, LPS treated mice experienced an over 300 fold increase in serum TNF-α levels that peaked at one hour post injection and decreased to basal levels by four hours post injection (FIG. 17a). In absence of PSA treatment, also IL-6 levels in the serum of LPS injected mice was detectable as early as 1 hour and continues to increase in expression by 4 hours, (FIG. 17b). Remarkably, mice that had been pre-treated with PSA had a significant reduction in serum levels of TNF-α and IL-6 at both time points (FIGS. 17a and 17b), indicating that PSA is able to prevent the early induction of inflammatory cytokines in response to LPS). Additionally, in absence of PSA treatment, splenomegaly occurs within three days of LPS injection and results from the recruitment of inflammatory cell types. Animals pre-treated with PSA, have smaller spleens and express lower levels of inflammatory cytokines at this site (data not shown and FIG. 17c).

This data demonstrates that PSA is capable of suppressing systemic inflammatory responses induced by a low dose administration of LPS.

### Example 12

### Parenteral Administration of PSA Results in TNF-a Modulation and Treatment Systemic Inflammation

Death occurring during endotoxic shock is a result of the elevated levels of inflammatory cytokines that occur within hours of the response to LPS. Indeed, blockage of the inflammatory mediator TNF-α completely rescues animals from LPS induced mortality. That PSA had such a dramatic effect on the levels of the cytokines expressed during low dose administration of LPS, suggested that PSA might prevent death associated with endotoxic shock. Applicants therefore administered high dose levels of LPS (500 that cause death with 24-96 hours and accessed both cytokine levels within the serum and monitored survival.

The results illustrated in FIGS. 17d and 17e, show that while animals that were administered PBS all die within 60 hours of administration of LPS, those animals that received PSA treatment have a significantly increased survival rate (FIG. 17d). Remarkably, while PBS animals have an over 3000 fold induction of TNF-α when administered LPS, those mice receiving PSA have very little TNF-a induction (FIG. 17e). These data demonstrate that PSA is able to suppress the systemic inflammatory response that ensues in response to LPS and is able to protect from septic shock.

As shown in the exemplary experiments of Example 7 PSA mediated protection from IBD is reliant on IL-10 production from a CD4+T lymphocyte. To determine whether IL-10 is required for protection from LPS induced death Applicants pretreated IL10 deficient animals with PBS or purified PSA and administer levels of LPS that would result in septic shock. The cytokine level and percentage survival were monitored.

The results illustrated in FIG. 18, show that consistent with previous data IL10-deficient animals were more sensitive to lower doses of LPS and TNF alpha levels continue increase (FIG. 18a). Interestingly, PSA treated animals have a drastic decrease in the levels of serum TNF-a in response to LPS that drops to negligible levels by 4 hours post LPS administration (FIG. 18a), indicating that decreased TNF-a levels by PSA is not dependent on the ability of PSA to induce IL-10. Strikingly, decreased IL-6 production by PSA is IL-10 dependent as levels are similar to PBS treated animals, indicating multiple mechanisms are employed by PSA to alleviate endotoxic shock (FIG. 18b). Finally, IL10 deficient mice receiving PSA are completely protected from LPS induced death (FIG. 18c).

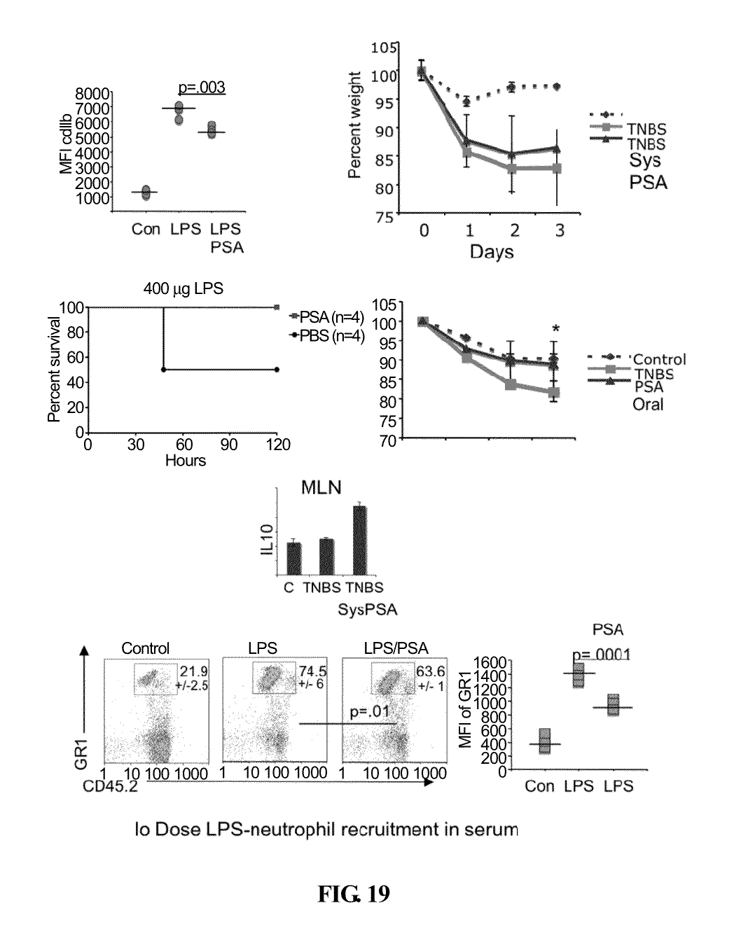

Additional experiments were performed to detect additional effects of PSA administration in connection with low dose LPS administration in mice. The results illustrated in FIG. 19 show that other effects of ZPS administration during low dose LPS administration include a reduction in CD11b and GR1 expression on the surface of neutrophils as well as reduced neutrophil recruitment in the blood.

Taken together, the data of this example and of example indicate that PSA is capable of blocking extra-intestinal disease and is expected to be a novel therapeutic agent to reduce systemic inflammation.

### Example 13

### PSA Increases Survival of Mice with Induced Colitis when Given Before or after Disease Induction

A first group of 8 week old Balb/c mice were orally gavaged with 50 μg of purified PSA three times every other day prior to colitis induction by rectal administration of 100 ml of 1.25% TNBS in 50% ethanol and every other day throughout the course of disease. A second group of 8 week old Balb/c mice were orally gavaged with 50 μg of PSA 24 hours post-colitis induction performed by rectal administration of 100 ml of 1.25% TNBS in 50% ethanol and every other day throughout the course of disease. Control mice were rectally administered 50% ethanol as a vehicle control. Each group had 6-11 mice.

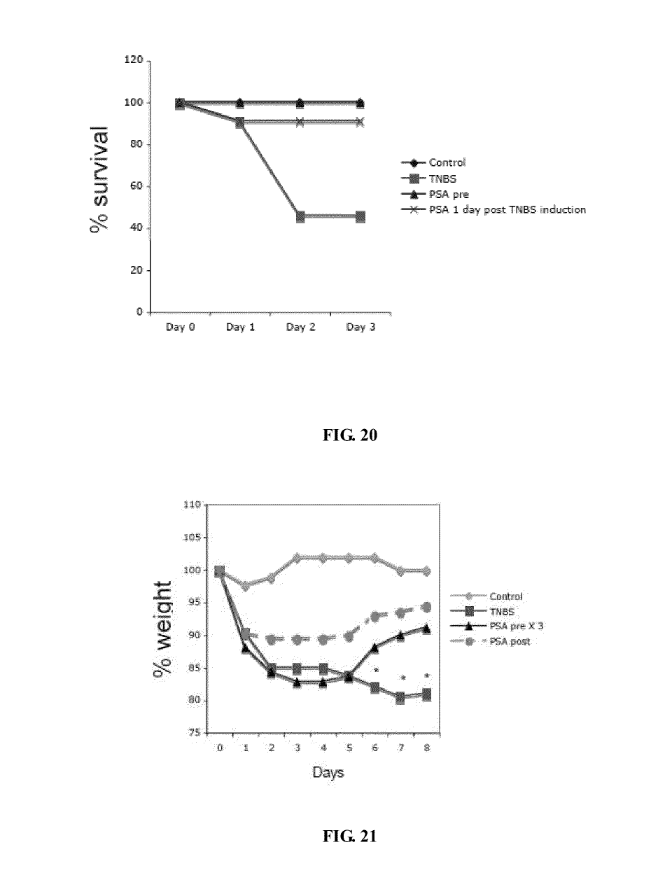

The percent survival was determined for each treatment and is illustrated in the diagram of FIG. 20. The diagram of FIG. 20 shows that only 45% of mice survive when treated with PBS alone (red square) while 100% of mice survive when pretreated with PSA and over 90% of mice survive when PSA is given as a treatment for disease, demonstrating that PSA can enhance the survival of mice even when disease has already begun.

### Example 14

### PSA can Prevent Disease Associated Weight Loss when Given Before or after Disease Induction

Balb/c mice were treated as described in Example 13 and were weighed on the day of TNBS induction (day 0) and every day thereafter at the same time of day.

The percentage variations in weight associated with the various treatments is illustrated in the diagram of FIG. 21 which further confirms that PSA can prevent disease associated weight loss when given before or after the induction. In particular, while, PBS treated-with TNBS induction (FIG. 21, squares)—showed a significant weight loss with respect to vehicle treated mice, with no induced TNBS (FIG. 21 diamonds)—, PSA treatment prior to TNBS induction (FIG. 21 triangles) and, PSA treatment after TNBS induction (FIG. 21 circles) significantly reduce the weight loss.

These results further confirm PSA ability to prevent and cure a disease associated with an imbalanced Th cell profile.

### Example 15

### PSA Administration Results in Treatment or Prevention of Experimental Colitis

Balb/c mice were treated as described in Example 13. Colons from mice were extracted 4 days post-TNBS induction and fixed in paraformaldehyde and embedded in paraffin. Sections of the colon were stained with H&E and examined by a blinded pathologist (Dr. Greg Lawson-University of California, Los Angeles) for signs of intestinal inflammation included epithelial hyperplasia, lymphocyte infiltration, and mucosal thickening.

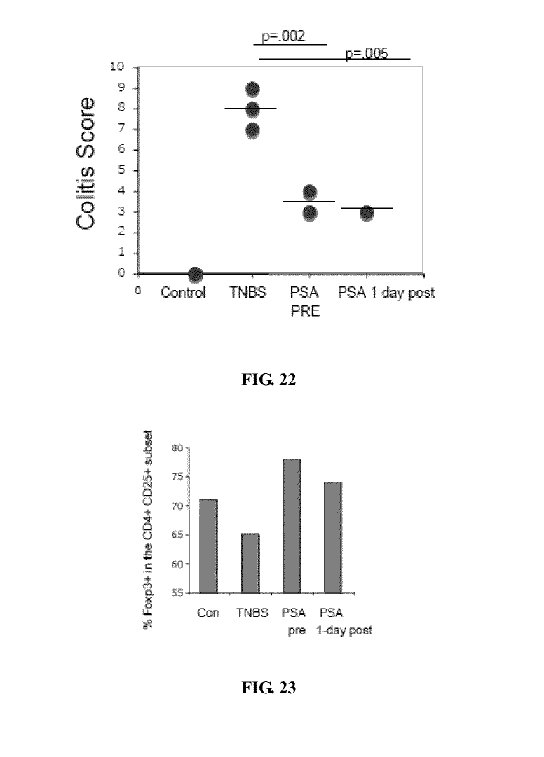

Increased colitis scores shown in the illustration of FIG. 22, indicate increasing severity of disease, thus further showing that PSA is able to prevent or treat on-going experimental intestinal inflammation. p values were calculated using a students t test.

Hence these results further confirm PSA ability to prevent and cure a disease associated with an imbalanced Th cell profile.

### Example 16

### PSA Administration Post-Disease Induction Increases the Percentage of Treg Cells in the Mesenteric Lymph Nodes

In previous experiments, it was shown that PSA can increase the number and percentage of Treg cells when given to mice before disease onset. Here a further series of experiments confirm that PSA can increase the percentage of Tregs cells within the MLNs when given to mice already suffering from disease, suggesting that the mechanism by which PSA can prevent or cure experimental colitis is through the expansion of a suppressive population of T cells.

MLNs were extracted from mice treated as described in Example 13. Cells were stained for CD4, CD25 and Foxp3 (as a marker of Treg cells).

The results illustrated in the diagram of FIG. 23 further confirm that PSA can prevent disease associated weight loss when given after the induction PSA administration post-disease induction increases the percentage of Treg cells in the mesenteric lymph nodes.

### Example 17

### PSA Promotes Inducible Foxp3+Tregs with Suppressive Activity Towards Inflammatory Responses

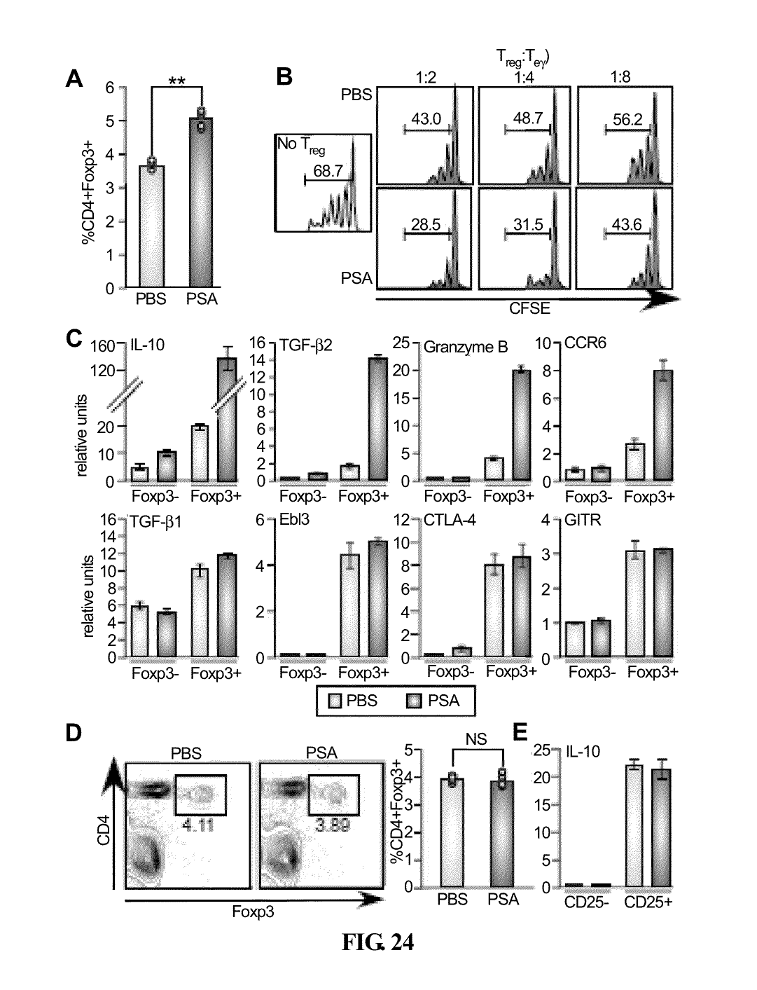

PSA was orally administered to mice and the CD4+CD25+Foxp3+ population of Tregs in the MLN was monitored. Mice treated with PSA displayed increased percentages of CD4+Foxp3+T cells compared to control mice (FIG. 24A). One of the primary functions of Treg cells is to suppress the activation and proliferation of inflammatory effector T cells. The suppressive properties of Tregs following PSA administration were determined by the addition of varying ratios of CD4+CD25+T cells purified from MLNs to naïve responder cells. Tregs isolated from PSA-treated animals have increased suppressive capacity compared to PBS-treated control animals (28.5% proliferating responder cells vs. 43.0% at a 1:2 Treg:Teff ratio) (FIG. 24B). These findings demonstrate that PSA induces the differentiation of functional Foxp3+Tregs with enhanced suppressive activity.

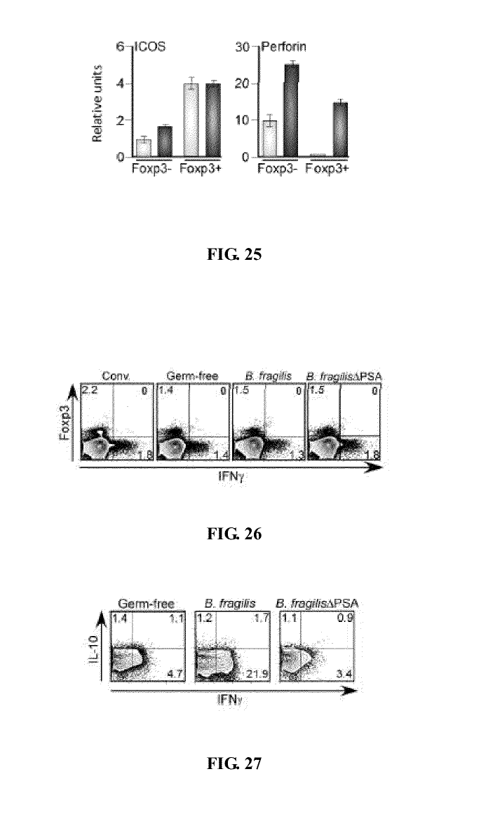

Various subsets of Tregs exist within the Foxp3+ and Foxp3− T cell populations; therefore, the expression of Treg-associated genes was analyzed to understand how PSA affects the development of Foxp3+Tregs. Foxp3-GFP mice were gavaged with purified PSA (or PBS control), and RNA was extracted from either CD4+Foxp3− or CD4+Foxp3+T cells from the MLNs following cell purification. As expected, gene expression in Foxp3− nd Foxp3+T cell subsets differed dramatically and included higher basal levels of IL-10, TGF-β2, GITR, ICOS, CTLA-4 and Ebi3 (subunit of IL-35) in Foxp3+T cells (FIG. 24,C and FIG. 25). Remarkably, PSA induces over 8-fold increased levels of IL-10 from CD4+Foxp3+Tregs than that expressed in PBS-treated cells, and had virtually no impact on CD4+Foxp3− T cells. Accordingly, while TGF-β2 expression in CD4+Foxp3− T cells was not altered, PSA elicited significant induction of TGF-β2 from Foxp3+Treg cells. Though Treg subsets that do not express Foxp3 have been described based on IL-10 and TGFβ production (Tr1 and Th3 cells, respectively)⁴⁴, PSA's effects are restricted to the Foxp3+Treg population. PSA treatment also significantly increases the transcription of granzyme B, perforin and CCR6 (a chemokine receptor associated with the migration of Treg cells) from Foxp3+Tregs (FIG. 24C and FIG. 25). It is important to note that PSA does not globally impact all Treg-derived cytokines, as expression of TGF-β1 and Ebi3 are not altered, demonstrating specificity for a distinct Treg profile. Furthermore, production of the natural Treg-associated surface receptors CTLA-4, GITR, and ICOS are not changed among Foxp3+ cells in response to PSA treatment (FIG. 24C and FIG. 25). Taken together, these data reveal that PSA activates inducible Foxp3+Tregs and identifies a TSA-specific′ gene expression program within Foxp3+Treg cells.

Colonization of germ-free animals with PSA-producing bacteria was previously reported to be able to induce production of the Th1 cytokine interferon-γ (IFNγ) among splenic CD4+T cells⁴⁰. To further investigate the lineage differentiation of immune cells directed by PSA, we examined animals for IFNγ expression among Foxp3+T cells. Intriguingly, IFNγ expression in the MLNs is found exclusively in the Foxp3-subset and is not affected by microbial colonization (FIG. 26). Furthermore, splenic CD4+T cells produce IFNγ only from Foxp3−/IL-10− T cells that is dependent on PSA expression (FIG. 27). These results demonstrate that there is a compartmental difference in the ability of PSA to induce a Th1 profile in the spleen, while promoting a tolerogenic immune environment in the gut consisting of CD4+Foxp3+IFNγ− Treg cells.

### Example 18

### PSA Expands Functional Foxp3+Tregs During Protection from Experimental Colitis

Intestinal inflammatory activation of innate and adaptive immune cells and secretion of inflammatory cytokines were not found under steady state conditions. Foxp3+Treg cell development was then examined during intestinal disease induced by TNBS. TNBS (2,4,6-trinitrobenzene sulfonic acid) treatment of animals results in gut inflammation which activates T cell responses; animals lose a significant amount of weight, display marked thickening of the colon, with lymphocyte infiltration and concomitant epithelial hyperplasia⁴⁵.

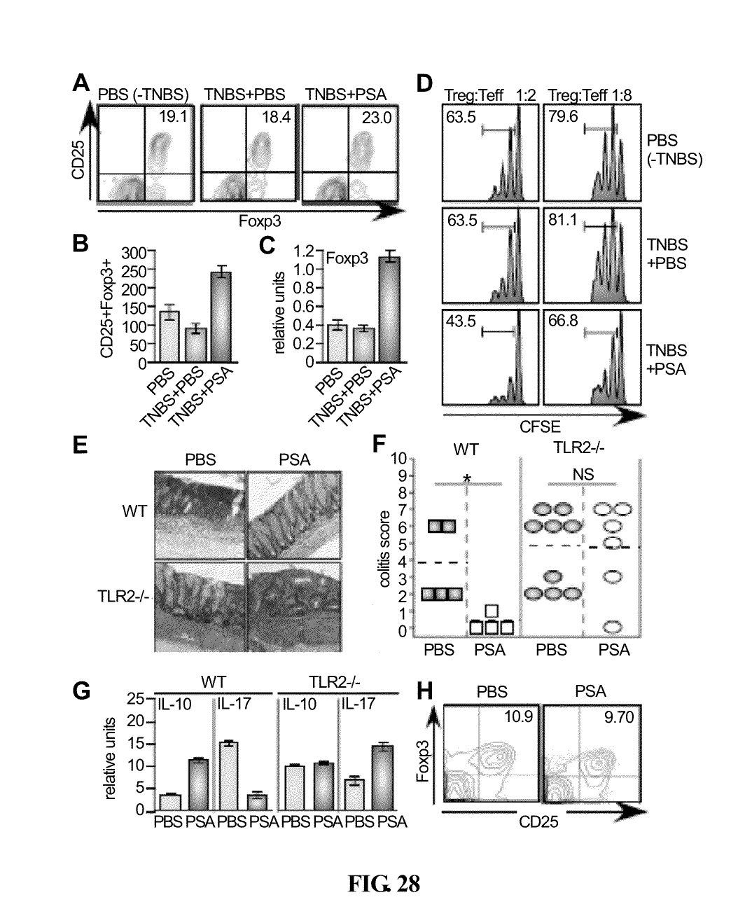

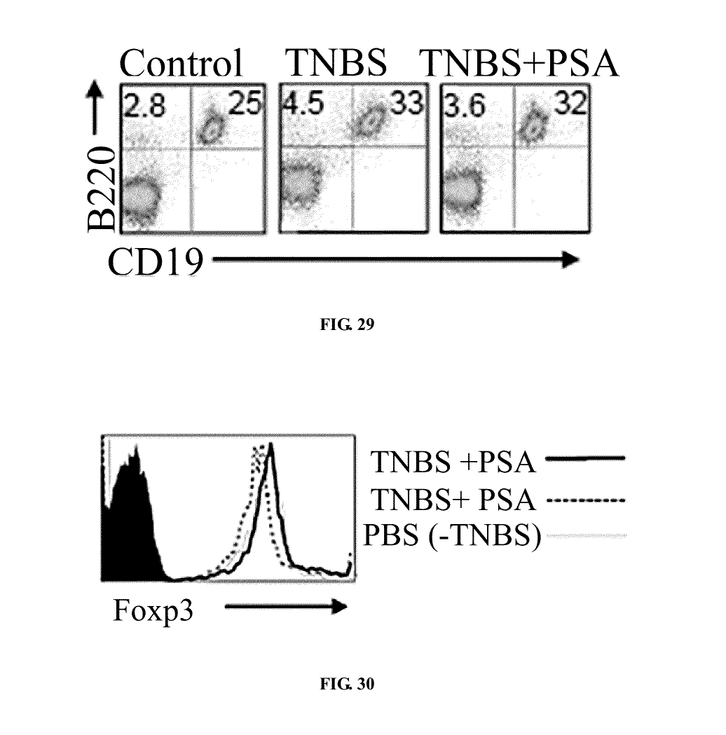

The results of these experiments are illustrated in FIGS. 28 to 30. As previously reported, disease was not evident in TNBS-treated animals that were treated with purified PSA⁴³. Vehicle treated (PBS; -TNBS) and PBS treated TNBS animals (TNBS+PBS) had a similar percentage of Foxp3+Treg cells within the CD4+CD25+T cells of the MLN (FIG. 28A). Consistent with PSA's anti-inflammatory properties, mice given PSA reproducibly had a 5-10% increase in the percentage of Foxp3+ cells within the CD4+CD25+ compartment of the MLNs (FIG. 28A). Additionally, the absolute number of CD4+CD25+Foxp3+ cells in the MLNs was significantly higher in PSA-treated mice when compared to PBS or non-colitic animals (FIG. 28B). PSA expansion of the Foxp3+Treg population is specific, as the percentage of B cells in the MLNs did not differ between PBS and PSA fed mice (FIG. 29).

Consistent with an increase in the percentage of Foxp3+ cells in PSA treated mice, there was an increase in the expression of the foxp3 transcript in MLNs (FIG. 28C). Furthermore, we also found that the Foxp3 expression was increased on a per cell basis in CD4+CD25+ cells during PSA-mediated protection from colitis (FIG. 30), demonstrating that PSA up-regulates proportional and cell-intrinsic Foxp3 expression. The suppressive capacity of Tregs during PSA-mediated protection from intestinal inflammation was determined by in vitro suppression assays.

As expected, proliferation was partially suppressed (proliferation of effector cells in the absence of Tregs was >than 90%) when Tregs from vehicle (PBS; −TNBS) or PBS-treated colitic mice (TNBS+PBS) were added to the culture (FIG. 28D). Notably however, Tregs isolated from the MLNs of PSA fed mice (TNBS+PSA) suppressed T cell proliferation to a significantly higher level than cells from untreated animals (43.5% proliferating responder cells vs. 63.5% at a 1:2 Treg:Teff ratio), demonstrating Tregs from animals protected from colitis by PSA have increased functional suppressive activity.

### Example 19

### PSA Treatment Cures Animals with Experimental Colitis

In various situations prophylactic therapies for IBD are unsuitable as there are no diagnostics that accurately predict disease development, and patients must be treated after onset of symptoms. The possibility that PSA could be efficacious for established colitis, potentiating its application as a treatment for established IBD was therefore investigated.

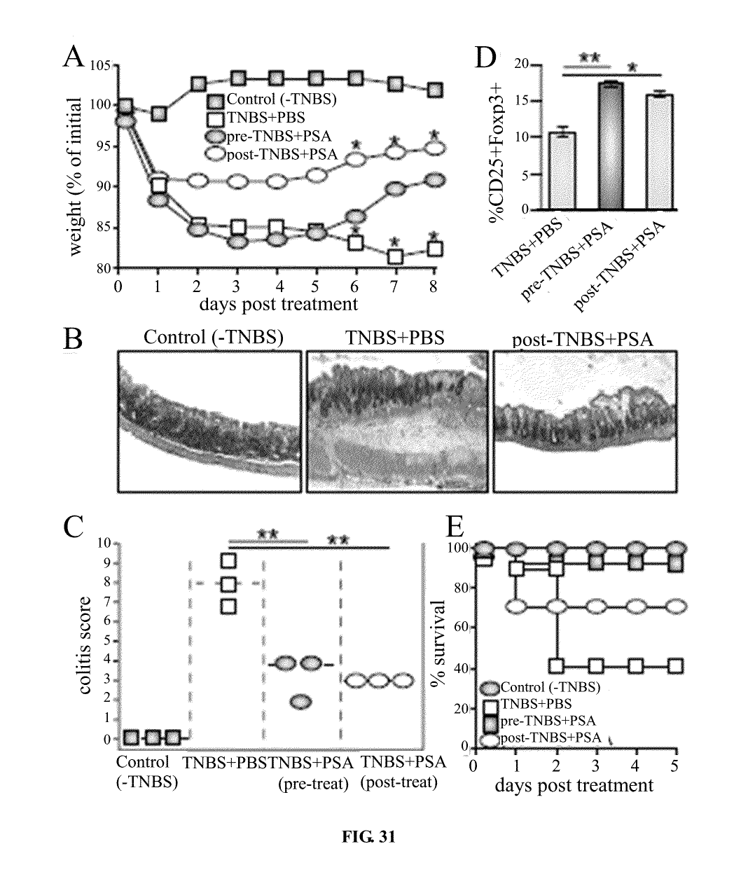

Animals were induced for TNBS colitis, and groups were treated with PBS prior to disease induction (TNBS+PBS) or PSA, either prior to disease induction (pre-TNBS+PSA) or following rectal TNBS administration (post-TNBS+PSA). PBS treated TNBS animals lost a significant amount of weight (approx. 20% weight loss) by 8 days (FIG. 31A).

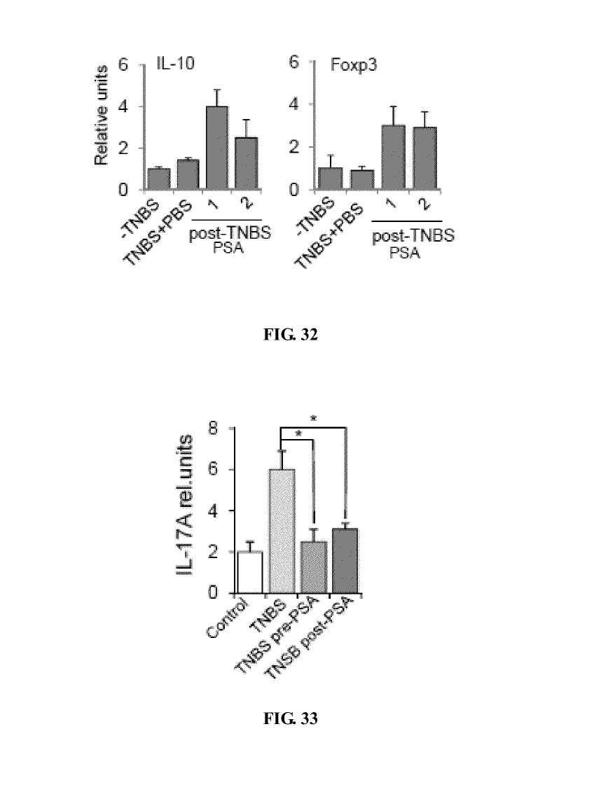

PSA treatment 1 day following onset of intestinal inflammation corrected weight loss equal to or better than PSA treatment prior to disease; treatment 2 days after colitis induction also provided protection (FIG. 32). Animals with colitis exhibited severe disease 5 days post-TNBS treatment which was not seen in animals treated with PSA post-TNBS (FIG. 31B).

Using a standard scoring system, animals treated with TNBS showed a high degree of disease (FIG. 31C). However, both pre- and post-treatment with PSA significantly prevented the development of colitis similarly. Animals administered PSA following the onset of disease showed increased IL-10 and Foxp3 expression in gut tissues (FIG. 32), and displayed a significant decrease in IL-17 levels during both protection and cure of colitis (FIG. 33). Accordingly, the percentage of CD4+CD25+Foxp3+ subset was significantly increased in PSA-treated animals compared to diseased animals (FIG. 31D). Rectal administration of high doses of TNBS causes a severe intestinal immune response that leads to mortality of animals. Remarkably, animals treated with PSA, even after the commencement of disease, were dramatically rescued from death compared to control treated animals (FIG. 31E).

Together our studies reveal that gut bacteria produce molecules that coordinate the development of inducible Foxp3+Tregs, and immunomodulation of Tregs by PSA can represent a novel approach to engendering mucosal tolerance as a therapy for intestinal inflammatory diseases.

### Example 20

### PSA Treatment Cures Animals Suffering from Endotoxic Shock

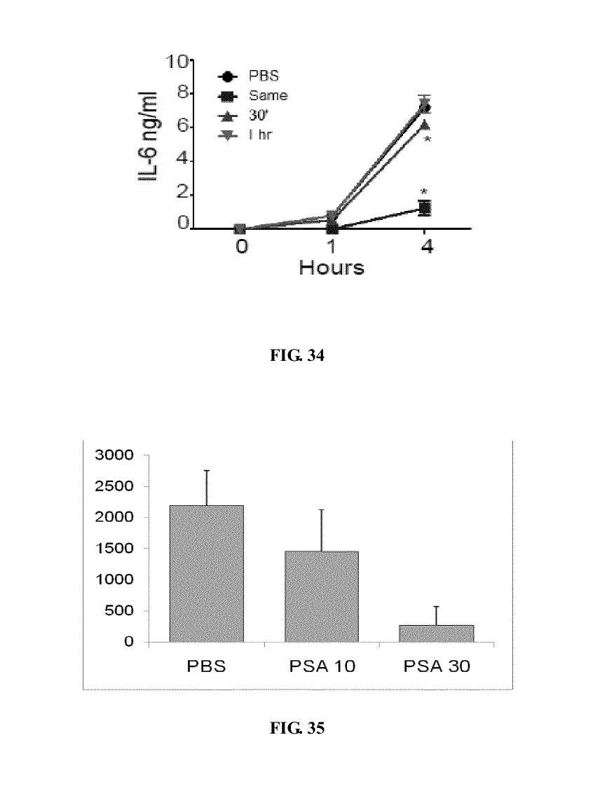

High doses of LPS (500 μg) were administered i.p. to Balb/c mice. 10 or 30 minutes post LPS administration, PSA was given retro-orbitally. 1 hour post-LPS, serum was collected and levels of 11-6 (FIG. 34) and TNF-α (FIG. 35) were determined. FIGS. 34 and 35 demonstrate that PSA can reduce inflammatory cytokines when administered after the systemic inflammatory stimulus (LPS) suggesting that PSA could be a therapy for endotoxic shock.

### Example 21

### PSA Treatment Cures Animals with Endotoxic Shock

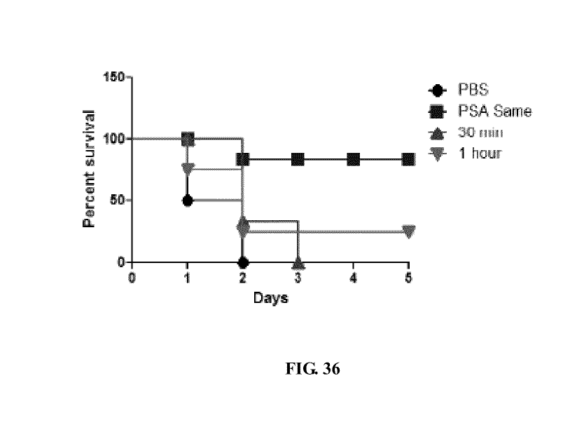

The results illustrated in FIG. 36 indicate that PSA can increase survival of mice already suffering from endotoxic shock. Mice were given 500 ug of LPS via i.p. injection. Animals were administered PSA systemically (retroorbital injection) 10 and 30 minutes after the administration of LPS. Survival of animals was determined.

The examples set forth above are provided to give those of ordinary skill in the art a complete disclosure and description of how to make and use the embodiments of the compounds compositions and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference.

Further, the hard copy of the sequence listing and the corresponding computer readable form submitted herewith are both incorporated herein by reference in their entireties.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing of the specific examples of appropriate materials and methods are described herein.

A number of embodiments of the disclosure have been described. Nevertheless, it will be understood that various modifications may be made without departing from the spirit and scope of the present disclosure. Accordingly, other embodiments are within the scope of the following claims.

### REFERENCES

1. Poxton, I. R., Brown, R., Sawyerr, A. & Ferguson, A. Mucosa-associated bacterial flora of the human colon. J Med Microbiol 46, 85-91 (1997)
2. Sellon, R. K. et al. Resident enteric bacteria are necessary for development of spontaneous colitis and immune system activation in interleukin-10-deficient mice. Infect Immun 66, 5224-31 (1998).
3. Elson, C. O. Commensal bacteria as targets in Crohn's disease. Gastroenterology 119, 254-7 (2000).
4. Sartor, R. B. Mechanisms of disease: pathogenesis of Crohn's disease and ulcerative colitis. Nat Clin Pract Gastroenterol Hepatol 3, 390-407 (2006).
5. Videla, S. et al. Role of intestinal microflora in chronic inflammation and ulceration of the rat colon. Gut 35, 1090-7 (1994).
6. Taurog, J. D. et al. The germfree state prevents development of gut and joint inflammatory disease in HLA-B27 transgenic rats. J Exp Med 180, 2359-64 (1994).
7. Kullberg, M. C. et al. Induction of colitis by a CD4+ T cell clone specific for a bacterial epitope. Proc Natl Acad Sci USA 100, 15830-5 (2003).
8. O'Hara, A. M. & Shanahan, F. The gut flora as a forgotten organ. EMBO Rep 7, 688-93 (2006).
9. Frank, D. N. et al. Molecular-phylogenetic characterization of microbial community imbalances in human inflammatory bowel diseases. Proc Natl Acad Sci USA 104, 13780-5 (2007).
10. Gill, S. R. et al. Metagenomic analysis of the human distal gut microbiome. Science 312, 1355-9 (2006).
11. Ley, R. E., Peterson, D. A. & Gordon, J. I. Ecological and evolutionary forces shaping microbial diversity in the human intestine. Cell 124, 837-48 (2006).
12. Smith, K., McCoy, K. D. & Macpherson, A. J. Use of axenic animals in studying the adaptation of mammals to their commensal intestinal microbiota. Semin Immunol (2006).
13. Mazmanian, S. K., Liu, C. H., Tzianabos, A. O. & Kasper, D. L. An immunomodulatory molecule of symbiotic bacteria directs maturation of the host immune system. Cell 122, 107-18 (2005).
14. Pamer, E. G. Immune responses to commensal and environmental microbes. Nat Immunol 8, 1173-8 (2007).
15. Dethlefsen, L., McFall-Ngai, M. & Relman, D. A. An ecological and evolutionary perspective on human-microbe mutualism and disease. Nature 449, 811-8 (2007).
16. Bell, E. B. Function of CD4 T cell subsets in vivo: expression of CD45R isoforms. Semin Immunol 4, 43-50 (1992).
17. Izcue, A., Coombes, J. L. & Powrie, F. Regulatory T cells suppress systemic and mucosal immune activation to control intestinal inflammation. Immunol Rev 212, 256-71 (2006).
18. Maloy, K. J. et al. CD4+CD25+ T(R) cells suppress innate immune pathology through cytokine-dependent mechanisms. J Exp Med 197, 111-9 (2003).
19. Cahill, R. J. et al. Inflammatory bowel disease: an immunity-mediated condition triggered by bacterial infection with Helicobacter hepaticus. Infect Immun 65, 3126-31 (1997).
20. Scheinin, T., Butler, D. M., Salway, F., Scallon, B. & Feldmann, M. Validation of the interleukin-10 knockout mouse model of colitis: antitumour necrosis factor-antibodies suppress the progression of colitis. Clin Exp Immunol 133, 38-43 (2003).
21. Kullberg, M. C. et al. Bacteria-triggered CD4(+) T regulatory cells suppress Helicobacter hepaticus-induced colitis. J Exp Med 196, 505-15 (2002).
22. Bregenholt, S. Cells and cytokines in the pathogenesis of inflammatory bowel disease: new insights from mouse T cell transfer models. Exp Clin Immunogenet 17, 115-29 (2000).
23. Powrie, F. & Maloy, K. J. Immunology. Regulating the regulators. Science 299, 1030-1 (2003).
24. Xavier, R. & Podolsky, D. K. Commensal flora: wolf in sheep's clothing. Gastroenterology 128, 1122-6 (2005).
25. Rutgeerts, P. et al. Infliximab for induction and maintenance therapy for ulcerative colitis. N Engl J Med 353, 2462-76 (2005).
26. Rakoff-Nahoum, S., Paglino, J., Eslami-Varzaneh, F., Edberg, S. & Medzhitov, R. Recognition of commensal microflora by toll-like receptors is required for intestinal homeostasis. Cell 118, 229-41 (2004).
27. Kullberg, M. C. et al. IL-23 plays a key role in Helicobacter hepaticus-induced T cell-dependent colitis. J Exp Med 203, 2485-94 (2006).
28. Hue, S. et al. Interleukin-23 drives innate and T cell-mediated intestinal inflammation. J Exp Med 203, 2473-83 (2006).
29. Tzianabos, A. O. et al. The capsular polysaccharide of Bacteroides fragilis comprises two ionically linked polysaccharides. J Biol Chem 267, 18230-5 (1992).
30. Elson, C. O. et al. Monoclonal anti-interleukin 23 reverses active colitis in a T cell-mediated model in mice. Gastroenterology 132, 2359-70 (2007).
31. Kuhn, R., Lohler, J., Rennick, D., Rajewsky, K. & Muller, W. Interleukin-10-deficient mice develop chronic enterocolitis. Cell 75, 263-74 (1993).
32. Asseman, C., Mauze, S., Leach, M. W., Coffman, R. L. & Powrie, F. An essential role for interleukin 10 in the function of regulatory T cells that inhibit intestinal inflammation. J Exp Med 190, 995-1004 (1999).
33. Groux, H. et al. A CD4+T-cell subset inhibits antigen-specific T-cell responses and prevents colitis. Nature 389, 737-42 (1997).
34. Strachan, D. P. Hay fever, hygiene, and household size. Bmj 299, 1259-60 (1989).
35. Bach, J. F. The effect of infections on susceptibility to autoimmune and allergic diseases. N Engl J Med 347, 911-20 (2002).
36. Liu, C. H., Lee, S. M., Vanlare, J. M., Kasper, D. L. & Mazmanian, S. K. Regulation of surface architecture by symbiotic bacteria mediates host colonization. Proc Natl Acad Sci USA 105, 3951-6 (2008).
37. Turnbaugh, P. J. et al. An obesity-associated gut microbiome with increased capacity for energy harvest. Nature 444, 1027-31 (2006).
38. Mazmanian, S. K. & Kasper, D. L. The love-hate relationship between bacterial polysaccharides and the host immune system. Nat Rev Immunol (2006).
39. Young, V. B. et al. In vitro and in vivo characterization of Helicobacter hepaticus cytolethal distending toxin mutants. Infect Immun 72, 2521-7 (2004).
40. Mazmanian, S. K., Liu, C. H., Tzianabos, A. O. & Kasper, D. L. An immunomodulatory molecule of symbiotic bacteria directs maturation of the host immune system. Cell 122, 107-18 (2005).
41. Rakoff-Nahoum, S., Paglino, J., Eslami-Varzaneh, F., Edberg, S. & Medzhitov, R. Recognition of commensal microflora by toll-like receptors is required for intestinal homeostasis. Cell 118, 229-41 (2004).

42. C Gerard, C Bruyns, A Marchant, D Abramowicz, P Vandenabeele, A Delvaux, W Fiers, M Goldman, and T Velu Interleukin 10 reduces the release of tumor necrosis factor and prevents lethality in experimental endotoxemia J. Exp. Med. 1993 177: 547-550
43. Sarkis K. Mazmanian, June L. Round & Dennis L. Kasper2, A microbial symbiosis factor prevents intestinal inflammatory disease Nature Vol 453, 29 May 2008, 620-625
44. O'Gana A, Vieira P L, Vieira P, & Goldfeld AE (2004) IL-10-producing and naturally occurring CD4+Tregs: limiting collateral damage. J Clin Invest 114(10):1372-1378
45. Neurath M, Fuss I, & Strober W (2000) TNBS-colitis. Int Rev Immunol 19(1):51-62.

1.-6. (canceled)

7. An anti-inflammatory pharmaceutical composition comprising:
a zwitterionic polysaccharide in an amount of from about 0.01 μg to about 1000 μg; and
a pharmaceutically acceptable vehicle.

8. The anti-inflammatory composition of claim 7, wherein the amount is between about 1 μg and about 100 μg.

9. The anti-inflammatory composition of claim 7, wherein the zwitterionic polysaccharide is a B fragilis capsular polysaccharide A (PSA) or polysaccharide B (PSB).

10. A method for treating a condition associated with a cytokine mediated inflammation in an individual the cytokine being at least one of IL-1, IL-6, TNF-α, IL-17, IL-21 and IL-23, the method comprising
orally or parenterally administering to the individual an amount of a purified zwitterionic polysaccharide effective to treat inflammatory bowel disease, dermatitis, arthritis, myasthenia gravis, Grave's disease, sclerosis, endotoxic shock or psoriasis.

11. The method of claim 10, wherein the cytokine is IL-17.

12. The method of claim 10, wherein the zwitterionic polysaccharide is a B. fragilis capsular polysaccharide A (PSA).

13. The method of claim 10, wherein the inflammatory bowel disease is Crohn's disease and/or ulcerative colitis.

14. A method for treating a condition associated with an imbalanced T-helper cell profile, the T-helper cell profile being at least one of Th17, the method
comprising orally or parenterally administering to the individual a pharmaceutical composition comprising a purified zwitterionic polysaccharide in an effective amount to treat rheumatoid arthritis, allograft rejection, systemic lupus erythematosus, tumorgenesis, multiple sclerosis, systemic sclerosis and chronic inflammatory bowel disease.

15. The method of claim 14, wherein the T-helper cell profile is a Th17 cell profile.


SEQUENCE LISTING (18 sequences)

SEQ ID NO: 1 - DNA, 15 bp, Artificial Sequence
  Synthetic Polynucleotide
gaaactgttactctg

SEQ ID NO: 2 - DNA, 17 bp, Artificial Sequence
  Synthetic Polynucleotide
tcaagctccccgaaggg

SEQ ID NO: 3 - DNA, 18 bp, Artificial Sequence
  Synthetic Polynucleotide
tatttgcgagaaggtgat

SEQ ID NO: 4 - DNA, 18 bp, Artificial Sequence
  Synthetic Polynucleotide
taaacgctttgctgctat

SEQ ID NO: 5 - DNA, 27 bp, Artificial Sequence
  Synthetic Polynucleotide
agctatgaatctactaagagagggaca

SEQ ID NO: 6 - DNA, 25 bp, Artificial Sequence
  Synthetic Polynucleotide
gtcctagtagggaggtgtgaagttg

SEQ ID NO: 7 - DNA, 20 bp, Artificial Sequence
  Synthetic Polynucleotide
ttaaggttctctcctctgaa

SEQ ID NO: 8 - DNA, 20 bp, Artificial Sequence
  Synthetic Polynucleotide
tagggagctaaattatccaa

SEQ ID NO: 9 - DNA, 20 bp, Artificial Sequence
  Synthetic Polynucleotide
acggcatggatctcaaagac

SEQ ID NO: 10 - DNA, 20 bp, Artificial Sequence
  Synthetic Polynucleotide
gtgggtgaggagcacgtagt

SEQ ID NO: 11 - DNA, 22 bp, Artificial Sequence
  Synthetic Polynucleotide
ctggacaacatactgctaaccg

SEQ ID NO: 12 - DNA, 22 bp, Artificial Sequence
  Synthetic Polynucleotide
gggcatcacttctaccaggtaa

SEQ ID NO: 13 - DNA, 18 bp, Artificial Sequence
  Synthetic Polynucleotide
ccgctgagagggcttcac

SEQ ID NO: 14 - DNA, 22 bp, Artificial Sequence
  Synthetic Polynucleotide
tgcaggagtaggccacattaca

SEQ ID NO: 15 - DNA, 24 bp, Artificial Sequence
  Synthetic Polynucleotide
atcctgaacttctatcagctccac

SEQ ID NO: 16 - DNA, 25 bp, Artificial Sequence
  Synthetic Polynucleotide
gcatttagctatgtgcttctgtttc

SEQ ID NO: 17 - DNA, 21 bp, Artificial Sequence
  Synthetic Polynucleotide
ctgttgctgctacccttgctt

SEQ ID NO: 18 - DNA, 21 bp, Artificial Sequence
  Synthetic Polynucleotide
cactcctggcaatcgagattc